(19) United States
(12) Patent Application Publication
(10) Pub. No.: US 2008/0305959 A1
(43) Pub. Date: Dec. 11, 2008

(54) Laser microdissection and microarray analysis of breast tumors reveal estrogen receptor related genes and pathways

(76) Inventors: Yixin Wang, San Diego, CA (US); Jack X. Yu, San Diego, CA (US); Yugiu Jiang, San Diego, CA (US); Fei Yang, San Diego, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/398,340

(22) Filed: Apr. 4, 2006

Related U.S. Application Data
(60) Provisional application No. 60/668,005, filed on Apr. 4, 2005.

(51) Int. Cl. C40B 30/04; C12Q 1/68; C07H 21/04; C40B 40/08

(52) U.S. Cl. 506/9; 435/6; 536/24.31; 506/17

(57) ABSTRACT

About 70% to 80% of breast cancers express estrogen receptor-α (ERα), and estrogens play important roles in the development and growth of hormone-dependent tumors. Together with lymph node metastasis, tumor size and histological grade, ER status is considered one of the prognostic factors in breast cancer, and an indicator for hormonal treatment. 147 genes and 112 genes with significant P-value and having significant differential expression between ER+ and ER− tumors were identified from the LCM data set and bulk tissue data set, respectively. 61 genes were found to be common in both data sets, while 85 genes were unique to the LCM data set and 51 genes were present only in the bulk tumor data set. Pathway analysis with the 85 genes using Gene Ontology suggested that genes involved in endocytosis, ceramide generation, Ras/ERK/Ark cascade, and JAT-STAT pathway may play roles related to ER. The gene profiling with LCM-captured tumor cells provides a unique approach to characterize and study epithelial tumor cells and to gain an insight into signaling pathways associated with ER.

### STATEMENT REGARDING FEDERALLY SPONSORED RESEARCH OR DEVELOPMENT

No government funds were used to make this invention.

### REFERENCE TO SEQUENCE LISTING, OR A COMPUTER PROGRAM LISTING COMPACT DISK APPENDIX

Reference to a “Sequence Listing,” appendix is specified.

### BACKGROUND OF THE INVENTION

About 70% to 80% of breast cancers express estrogen receptor-α (ERα), and estrogens play important roles in the development and growth of hormone-dependent tumors. Together with lymph node metastasis, tumor size and histological grade, ER status is considered one of the prognostic factors in breast cancer, and an indicator for hormonal treatment. Breast cancer is the most frequently diagnosed cancer and the second leading cause of cancer death among women in the US. Estrogens play important roles in the growth and differentiation of normal mammary gland, as well as in the development and progression of breast carcinoma. Estrogens regulate gene expression via ERα, which is expressed in about 70% to 80% of all breast cancers. Parl (2000). In current clinical practice, the presence of ER is a marker for selecting hormonal or aromatase inhibitors treatment in patients with primary or recurred breast cancers. Mokbel (2003). Extensive studies have described that ERs are ligand-activated transcription factors that mediate the pleiotropic effects of the steroid hormone estrogen on the growth, development and maintenance of several target tissues. Moggs et al. (2001). Mechanisms by which estrogen receptor mediates the transactivation of gene expression are complex. Hall et al. (2001) summarized the following four pathways: 1) classical ligand-dependent pathway in which the ER complex regulates gene transcription through its interaction with ERE consensus DNA sequences; 2) ligand-independent pathway in which growth factors and their tyrosine kinase receptors may activate ER and increase the expression of ER target genes in the absence of estrogen; 3) DNA binding-independent pathway in which induction of gene regulation by ER complex is through interactions with no ERE-like promoter elements such as AP1, SP1 and CREs; and 4) cell-surface (nongenomic) signaling in which estrogen activates a putative membrane-associated binding site that generate rapid tissue responses. However, the details of the estrogen effect on downstream gene targets, the role of cofactors, and cross talk between other signaling pathways are still largely unknown.

Gene-expression profiling technologies have empowered researchers to address complex questions in tumor biology. Many studies have shown the distinct patterns of gene expression related to ER status in breast cancer, and identified genes related to ER signaling. WO 2004/079014; West et al. (2001); Gruvberger et al. (2001); and Sotiriou et al. (2003). However, most data were based on expression of mRNAs isolated from tumor masses, which constitute various cell populations such as stroma cells, fibroblasts and lymphocytes, in addition to cancer cells. Moreover, the proportion of tumor cells in clinical samples varies significantly. These issues may compromise the gene expression data associated with ER that is expressed specifically on the epithelial cells. Laser capture microdissection (LCM) (Emmert-Buck et al. 1996), a technique that procures histologically homogenous cell populations, has recently been successfully used in combination with DNA microarray technologies in studies of various types of tumors (Luo et al. 1999; Matsui et al. 2003; Yim et al. 2003; and Nakamura et al. 2004), including breast cancer for which genes were identified in association with tumor progression and metastasis. Ma et al. (2002); Seth et al. (2003); and Nishidate et al. (2004).

### SUMMARY OF THE INVENTION

The present invention provides a method of determining estrogen receptor expression status by obtaining a bulk tissue tumor sample from a breast cancer patient; and measuring the expression levels in the sample of genes encoding mRNA: i. corresponding to SEQ ID Nos listed in Table 2 or 3; or ii. recognized by the probe sets psids corresponding to SEQ ID Nos listed in Table 2 or 3 where the gene expression levels above or below pre-determined cut-off levels are indicative of estrogen receptor expression status.

The present invention provides a method of determining estrogen receptor expression status by obtaining a microscopically isolated tumor sample from a breast cancer patient; and measuring the expression levels in the sample of genes those encoding mRNA: i. corresponding to SEQ ID Nos listed in Table 2 or 4; or ii. recognized by the probe sets psids corresponding to SEQ ID Nos listed in Table 2 or 4 where the gene expression levels above or below pre-determined cut-off levels are indicative of estrogen receptor expression status.

The present invention provides a method of determining breast cancer patient treatment protocol by obtaining a bulk tissue tumor sample from a breast cancer patient; and measuring the expression levels in the sample of genes those encoding mRNA: i. corresponding to SEQ ID Nos listed in Table 2 or 3; or ii. recognized by the probe sets psids corresponding to SEQ ID Nos listed in Table 2 or 3 where the gene expression levels above or below pre-determined cut-off levels are sufficiently indicative of risk of recurrence to enable a physician to determine the degree and type of therapy recommended to prevent recurrence.

The present invention provides a method of determining breast cancer patient treatment protocol by obtaining a microscopically isolated tumor sample from a breast cancer patient; and measuring the expression levels in the sample of genes those encoding mRNA: i. corresponding to SEQ ID Nos listed in Table 2 or 4; or ii. recognized by the probe sets psids corresponding to SEQ ID Nos listed in Table 2 or 4 where the gene expression levels above or below pre-determined cut-off levels are sufficiently indicative of risk of recurrence to enable a physician to determine the degree and type of therapy recommended to prevent recurrence.

The present invention provides a method of treating a breast cancer patient by obtaining a bulk tissue tumor sample from a breast cancer patient; and measuring the expression levels in the sample of genes those encoding mRNA: i. corresponding to SEQ ID Nos listed in Table 2 or 3; or ii. recognized by the probe sets psids corresponding to SEQ ID Nos listed in Table 2 or 3 and; treating the patient with adjuvant therapy if they are a high risk patient.

The present invention provides a method of treating a breast cancer patient by obtaining a microscopically isolated tumor sample from a breast cancer patient; and measuring the expression levels in the sample of genes those encoding mRNA: i. corresponding to SEQ ID Nos listed in Table 2 or 4; or ii. recognized by the probe sets psids corresponding to SEQ ID Nos listed in Table 2 or 4 and; treating the patient with adjuvant therapy if they are a high risk patient.

The present invention provides a composition comprising at least one probe set the SEQ ID NOs: listed in Table 2, 3 and/or 4.

The present invention provides a kit for conducting an assay to determine estrogen receptor expression status a biological sample comprising: materials for detecting isolated nucleic acid sequences, their complements, or portions thereof of a combination of genes those encoding mRNA corresponding to the SEQ ID NOs: listed in Table 2, 3 and/or 4.

The present invention provides articles for assessing breast cancer status comprising: materials for detecting isolated nucleic acid sequences, their complements, or portions thereof of a combination of genes those encoding mRNA corresponding to the SEQ ID NOs: listed in Table 2, 3 and/or 4.

The present invention provides a microarray or gene chip for performing the method of any one of the methods described herein.

The present invention provides a diagnostic/prognostic portfolio comprising isolated nucleic acid sequences, their complements, or portions thereof of a combination of genes those encoding mRNA corresponding to the SEQ ID NOs: listed in Table 2, 3 and/or 4.

### BRIEF DESCRIPTION OF THE DRAWINGS

FIG. 1 depicts the comparison of expression intensities of 21 consecutively expressed housekeeping genes between the bulk tumor data set and the LCM-procured sample data set.

FIG. 2 depicts unsupervised two-dimensional hierarchical clustering analysis of the global gene expression data using Gene Spring software. A filter was applied to include genes that had “present” calls in at least two samples. Each horizontal row represents a gene, and each vertical column corresponds to a sample. Red or green color indicates a transcription level above or below the median expression of the genes across all samples. Blue bars represent the LCM sample data and yellow bars represent the bulk tumor data. ER status of the patients determined by ligand binding assay was represented as darker green blocks for ER+ patients and light green blocks for ER− patients. Bars A, B, C and D represent major sub-groups within the LCM and bulk tissue clusters.

FIG. 3 depicts pathway analyses of differentially expressed genes between ER+ subgroup and ER− subgroup. The categories that had at least 10 genes on the chip were used for following pathway analyses. A list of genes that were selected from data analysis was mapped to the GO Biological Process categories. Then hypergeometric distribution probability of the genes was calculated for each category. The categories that had a p-value less than 0.05 and at least two genes were considered over-represented in the selected gene list. 3A represents the pie chart of the number of genes designated to the three following categories: common in both LCM data set and bulk tumor data set; unique to the LCM sample data set; unique to the bulk tumor data set. 3B listed pathways that were identified with the common gene list, 3C shows the significant pathways with genes that are unique to the LCM data set, and 3D represents the pathways that are unique to the bulk tumor data set. P-values are specified beside bars.

### DETAILED DESCRIPTION

To investigate genes and pathways that are associated with ER status and epithelial cells in breast tumors, we applied laser capture microdissection (LCM) technology to capture epithelial tumor cells from 28 lymph node negative breast tumor samples, in which 17 patients had ER+ tumors, and 11 patients had ER− tumors. Gene expression profiles were analyzed on Affymetrix Hu133A GeneChips. Meanwhile, gene profiles using total RNA isolated from bulk tumors of the same 28 patients were also generated. 146 genes and 112 genes with significant P-value and having significant differential expression between ER+ and ER− tumors were identified from the LCM data set and bulk tissue data set, respectively. 61 genes were found to be common in both data sets, while 85 genes were unique to the LCM data set and 51 genes were present only in the bulk tumor data set. Pathway analysis with the 85 genes using Gene Ontology suggested that genes involved in endocytosis, ceramide generation, Ras/ERK/Ark cascade, and JAT-STAT pathway may play roles related to ER. The gene profiling with LCM-captured tumor cells provides a unique approach to study epithelial tumor cells and to gain an insight into signaling pathways associated with ER.

The present invention provides a method of determining estrogen receptor expression status by obtaining a bulk tissue tumor sample from a breast cancer patient; and measuring the expression levels in the sample of genes encoding mRNA: i. corresponding to SEQ ID Nos listed in Table 2 or 3; or ii. recognized by the probe sets psids corresponding to SEQ ID Nos listed in Table 2 or 3 where the gene expression levels above or below pre-determined cut-off levels are indicative of estrogen receptor expression status.

The sample can be obtained from a primary tumor such as from a biopsy or a surgical specimen. The method can further include measuring the expression level of at least one gene constitutively expressed in the sample. In one embodiment, the method yields a result where the specificity is at least about 40% and the sensitivity is at least at least about 90%. In another embodiment, the expression pattern of the genes is compared to an expression pattern indicative of a relapse patient. The comparison of expression patterns can be conducted with pattern recognition methods such as a Cox's proportional hazards analysis.

In one embodiment, the pre-determined cut-off levels are at least 1.5-fold over- or under-expression in the sample relative to benign cells or normal tissue. In another embodiment, the pre-determined cut-off levels have at least a statistically significant p-value over- or under-expression in the sample having metastatic cells relative to benign cells or normal tissue. Preferably, the p-value is less than 0.05.

In one embodiment, gene expression is measured on a microarray or gene chip such as a cDNA array or an oligonucleotide array. The microarray or gene chip can further contain one or more internal control reagents. In one embodiment, gene expression is determined by nucleic acid amplification conducted by polymerase chain reaction (PCR) of RNA extracted from the sample. PCR can be by reverse transcription polymerase chain reaction (RT-PCR) and can contain one or more internal control reagents. In one embodiment, gene expression is detected by measuring or detecting a protein encoded by the gene such as by an antibody specific to the protein. In one embodiment, gene expression is detected by measuring a characteristic of the gene such as DNA amplification, methylation, mutation and allelic variation.

The present invention provides a method of determining estrogen receptor expression status by obtaining a microscopically isolated tumor sample from a breast cancer patient; and measuring the expression levels in the sample of genes those encoding mRNA: i. corresponding to SEQ ID Nos listed in Table 2 or 4; or ii. recognized by the probe sets psids corresponding to SEQ ID Nos listed in Table 2 or 4 where the gene expression levels above or below pre-determined cut-off levels are indicative of estrogen receptor expression status.

The sample can be obtained from a primary tumor. The microscopic isolation can be, for instance, by laser capture microdissection. The method can further include measuring the expression level of at least one gene constitutively expressed in the sample. In one embodiment, the method yields a result where the specificity is at least about 40% and the sensitivity is at least at least about 90%. In another embodiment, the expression pattern of the genes is compared to an expression pattern indicative of a relapse patient. The comparison of expression patterns can be conducted with pattern recognition methods such as a Cox's proportional hazards analysis.

In one embodiment, the pre-determined cut-off levels are at least 1.5-fold over- or under-expression in the sample relative to benign cells or normal tissue. In another embodiment, the pre-determined cut-off-levels have at least a statistically significant p-value over- or under-expression in the sample having metastatic cells relative to benign cells or normal tissue. Preferably, the p-value is less than 0.05.

In one embodiment, gene expression is measured on a microarray or gene chip such as a cDNA array or an oligonucleotide array. The microarray or gene chip can further contain one or more internal control reagents. In one embodiment, gene expression is determined by nucleic acid amplification conducted by polymerase chain reaction (PCR) of RNA extracted from the sample. PCR can be by reverse transcription polymerase chain reaction (RT-PCR) and can contain one or more internal control reagents. In one embodiment, gene expression is detected by measuring or detecting a protein encoded by the gene such as by an antibody specific to the protein. In one embodiment, gene expression is detected by measuring a characteristic of the gene such as DNA amplification, methylation, mutation and allelic variation.

The present invention provides a method of determining breast cancer patient treatment protocol by obtaining a bulk tissue tumor sample from a breast cancer patient; and measuring the expression levels in the sample of genes those encoding mRNA: i. corresponding to SEQ ID Nos listed in Table 2 or 3; or ii. recognized by the probe sets psids corresponding to SEQ ID Nos listed in Table 2 or 3 where the gene expression levels above or below pre-determined cut-off levels are sufficiently indicative of risk of recurrence to enable a physician to determine the degree and type of therapy recommended to prevent recurrence.

The sample can be obtained from a primary tumor such as from a biopsy or a surgical specimen. The method can further include measuring the expression level of at least one gene constitutively expressed in the sample. In one embodiment, the method yields a result where the specificity is at least about 40% and the sensitivity is at least at least about 90%. In another embodiment, the expression pattern of the genes is compared to an expression pattern indicative of a relapse patient. The comparison of expression patterns can be conducted with pattern recognition methods such as a Cox's proportional hazards analysis.

In one embodiment, the pre-determined cut-off levels are at least 1.5-fold over- or under-expression in the sample relative to benign cells or normal tissue. In another embodiment, the pre-determined cut-off levels have at least a statistically significant p-value over- or under-expression in the sample having metastatic cells relative to benign cells or normal tissue. Preferably, the p-value is less than 0.05.

In one embodiment, gene expression is measured on a microarray or gene chip such as a cDNA array or an oligonucleotide array. The microarray or gene chip can further contain one or more internal control reagents. In one embodiment, gene expression is determined by nucleic acid amplification conducted by polymerase chain reaction (PCR) of RNA extracted from the sample. PCR can be by reverse transcription polymerase chain reaction (RT-PCR) and can contain one or more internal control reagents. In one embodiment, gene expression is detected by measuring or detecting a protein encoded by the gene such as by an antibody specific to the protein. In one embodiment, gene expression is detected by measuring a characteristic of the gene such as DNA amplification, methylation, mutation and allelic variation.

The present invention provides a method of determining breast cancer patient treatment protocol by obtaining a microscopically isolated tumor sample from a breast cancer patient; and measuring the expression levels in the sample of genes those encoding mRNA: i. corresponding to SEQ ID Nos listed in Table 2 or 4; or ii. recognized by the probe sets psids corresponding to SEQ ID Nos listed in Table 2 or 4 where the gene expression levels above or below pre-determined cut-off levels are sufficiently indicative of risk of recurrence to enable a physician to determine the degree and type of therapy recommended to prevent recurrence.

The sample can be obtained from a primary tumor. The microscopic isolation can be, for instance, by laser capture microdissection. The method can further include measuring the expression level of at least one gene constitutively expressed in the sample. In one embodiment, the method yields a result where the specificity is at least about 40% and the sensitivity is at least at least about 90%. In another embodiment, the expression pattern of the genes is compared to an expression pattern indicative of a relapse patient. The comparison of expression patterns can be conducted with pattern recognition methods such as a Cox's proportional hazards analysis.

In one embodiment, the pre-determined cut-off levels are at least 1.5-fold over- or under-expression in the sample relative to benign cells or normal tissue. In another embodiment, the pre-determined cut-off levels have at least a statistically significant p-value over- or under-expression in the sample having metastatic cells relative to benign cells or normal tissue. Preferably, the p-value is less than 0.05.

In one embodiment, gene expression is measured on a microarray or gene chip such as a cDNA array or an oligonucleotide array. The microarray or gene chip can further contain one or more internal control reagents. In one embodiment, gene expression is determined by nucleic acid amplification conducted by polymerase chain reaction (PCR) of RNA extracted from the sample. PCR can be by reverse transcription polymerase chain reaction (RT-PCR) and can contain one or more internal control reagents. In one embodiment, gene expression is detected by measuring or detecting a protein encoded by the gene such as by an antibody specific to the protein. In one embodiment, gene expression is detected by measuring a characteristic of the gene such as DNA amplification, methylation, mutation and allelic variation.

The present invention provides a method of treating a breast cancer patient by obtaining a bulk tissue tumor sample from a breast cancer patient; and measuring the expression levels in the sample of genes those encoding mRNA: i. corresponding to SEQ ID Nos listed in Table 2 or 3; or ii. recognized by the probe sets psids corresponding to SEQ ID Nos listed in Table 2 or 3 and; treating the patient with adjuvant therapy if they are a high risk patient.

The sample can be obtained from a primary tumor such as from a biopsy or a surgical specimen. The method can further include measuring the expression level of at least one gene constitutively expressed in the sample. In one embodiment, the method yields a result where the specificity is at least about 40% and the sensitivity is at least at least about 90%. In another embodiment, the expression pattern of the genes is compared to an expression pattern indicative of a relapse patient. The comparison of expression patterns can be conducted with pattern recognition methods such as a Cox's proportional hazards analysis.

In one embodiment, the pre-determined cut-off levels are at least 1.5-fold over- or under-expression in the sample relative to benign cells or normal tissue. In another embodiment, the pre-determined cut-off levels have at least a statistically significant p-value over- or under-expression in the sample having metastatic cells relative to benign cells or normal tissue. Preferably, the p-value is less than 0.05.

In one embodiment, gene expression is measured on a microarray or gene chip such as a cDNA array or an oligonucleotide array. The microarray or gene chip can further contain one or more internal control reagents. In one embodiment, gene expression is determined by nucleic acid amplification conducted by polymerase chain reaction (PCR) of RNA extracted from the sample. PCR can be by reverse transcription polymerase chain reaction (RT-PCR) and can contain one or more internal control reagents. In one embodiment, gene expression is detected by measuring or detecting a protein encoded by the gene such as by an antibody specific to the protein. In one embodiment, gene expression is detected by measuring a characteristic of the gene such as DNA amplification, methylation, mutation and allelic variation.

The present invention provides a method of treating a breast cancer patient by obtaining a microscopically isolated tumor sample from a breast cancer patient; and measuring the expression levels in the sample of genes those encoding mRNA: i. corresponding to SEQ ID Nos listed in Table 2 or 4; or ii. recognized by the probe sets psids corresponding to SEQ ID Nos listed in Table 2 or 4 and; treating the patient with adjuvant therapy if they are a high risk patient.

The sample can be obtained from a primary tumor. The microscopic isolation can be, for instance, by laser capture microdissection. The method can further include measuring the expression level of at least one gene constitutively expressed in the sample. In one embodiment, the method yields a result where the specificity is at least about 40% and the sensitivity is at least at least about 90%. In another embodiment, the expression pattern of the genes is compared to an expression pattern indicative of a relapse patient. The comparison of expression patterns can be conducted with pattern recognition methods such as a Cox's proportional hazards analysis.

In one embodiment, the pre-determined cut-off levels are at least 1.5-fold over- or under-expression in the sample relative to benign cells or normal tissue. In another embodiment, the pre-determined cut-off levels have at least a statistically significant p-value over- or under-expression in the sample having metastatic cells relative to benign cells or normal tissue. Preferably, the p-value is less than 0.05.

In one embodiment, gene expression is measured on a microarray or gene chip such as a cDNA array or an oligonucleotide array. The microarray or gene chip can further contain one or more internal control reagents. In one embodiment, gene expression is determined by nucleic acid amplification conducted by polymerase chain reaction (PCR) of RNA extracted from the sample. PCR can be by reverse transcription polymerase chain reaction (RT-PCR) and can contain one or more internal control reagents. In one embodiment, gene expression is detected by measuring or detecting a protein encoded by the gene such as by an antibody specific to the protein. In one embodiment, gene expression is detected by measuring a characteristic of the gene such as DNA amplification, methylation, mutation and allelic variation.

The present invention provides a composition comprising at least one probe set the SEQ ID NOs: listed in Table 2, 3 and/or 4 such as a kit, article, microarray, etc.

The present invention provides a kit for conducting an assay to determine estrogen receptor expression status a biological sample comprising: materials for detecting isolated nucleic acid sequences, their complements, or portions thereof of a combination of genes those encoding mRNA corresponding to the SEQ ID NOs: listed in Table 2, 3 and/or 4. In one embodiment, the SEQ ID NOs. are those in Table 2 and/or 3. In another embodiment, the SEQ ID NOs. are listed in Table 2 and/or 4. The kit can further contain reagents for conducting a microarray analysis such as a medium through which said nucleic acid sequences, their complements, or portions thereof are assayed.

The present invention provides articles for assessing breast cancer status comprising: materials for detecting isolated nucleic acid sequences, their complements, or portions thereof of a combination of genes those encoding mRNA corresponding to the SEQ ID NOs: listed in Table 2, 3 and/or 4. In one embodiment, the SEQ ID NOs. are those in Table 2 and/or 3. In another embodiment, the SEQ ID NOs. are listed in Table 2 and/or 4. The articles can further contain reagents for conducting a microarray analysis such as a medium through which said nucleic acid sequences, their complements, or portions thereof are assayed.

The present invention provides a microarray or gene chip for performing the method of any one of the methods described herein. The microarray can contain isolated nucleic acid sequences, their complements, or portions thereof of a combination of genes those encoding mRNA corresponding to the SEQ ID NOs: listed in Table 2, 3 and/or 4. The microarray can further contain a cDNA array or an oligonucleotide array. The microarray can further contain or more internal control reagents.

The present invention provides a diagnostic/prognostic portfolio comprising isolated nucleic acid sequences, their complements, or portions thereof of a combination of genes those encoding mRNA corresponding to the SEQ ID NOs: listed in Table 2, 3 and/or 4.

Gene expression profiling using microscopically isolated breast tumor cells has not only identified differentially expressed genes related to ER status, but provides new information regarding pathways associated with estrogen signaling. The elucidation of the functional and clinical significance of these genes is also useful in determining breast tumor development by correlating expression levels of the identified genes with tumor progression or stage. The identification of breast epithelia specific genes further provides advantages in drug discovery for breast cancers by monitoring expression levels of the identified genes in tissue or in vitro expression systems in response to the presence or a drug or other substance.

The mere presence or absence of particular nucleic acid sequences in a tissue sample has only rarely been found to have diagnostic or prognostic value. Information about the expression of various proteins, peptides or mRNA, on the other hand, is increasingly viewed as important. The mere presence of nucleic acid sequences having the potential to express proteins, peptides, or mRNA (such sequences referred to as “genes”) within the genome by itself is not determinative of whether a protein, peptide, or mRNA is expressed in a given cell. Whether or not a given gene capable of expressing proteins, peptides, or mRNA does so and to what extent such expression occurs, if at all, is determined by a variety of complex factors. Irrespective of difficulties in understanding and assessing these factors, assaying gene expression can provide useful information about the occurrence of important events such as tumorogenesis, metastasis, apoptosis, and other clinically relevant phenomena. Relative indications of the degree to which genes are active or inactive can be found in gene expression profiles. The gene expression profiles of this invention are used to provide a prognosis and treat patients for breast cancer.

Sample preparation requires the collection of patient samples. Patient samples used in the inventive method are those that are suspected of containing diseased cells such as epithelial cells taken from the primary tumor in a breast sample. Samples taken from surgical margins are also preferred. Most preferably, however, the sample is taken from a lymph node obtained from a breast cancer surgery. Laser Capture Microdissection (LCM) technology is one way to select the cells to be studied, minimizing variability caused by cell type heterogeneity. Consequently, moderate or small changes in gene expression between normal and cancerous cells can be readily detected. Samples can also comprise circulating epithelial cells extracted from peripheral blood. These can be obtained according to a number of methods but the most preferred method is the magnetic separation technique described in U.S. Pat. No. 6,136,182. Once the sample containing the cells of interest has been obtained, RNA is extracted and amplified and a gene expression profile is obtained, preferably via micro-array, for genes in the appropriate portfolios.

Preferred methods for establishing gene expression profiles include determining the amount of RNA that is produced by a gene that can code for a protein or peptide. This is accomplished by RT-PCR, competitive RT-PCR, real time RT-PCR, differential display RT-PCR, Northern Blot analysis and other related tests. While it is possible to conduct these techniques using individual PCR reactions, it is best to amplify complementary DNA (cDNA) or complementary RNA (cRNA) produced from mRNA and analyze it via microarray. A number of different array configurations and methods for their production are known to those of skill in the art and are described in U.S. patents such as: U.S. Pat. Nos. 5,445,934; 5,532,128; 5,556,752; 5,242,974; 5,384,261; 5,405,783; 5,412,087; 5,424,186; 5,429,807; 5,436,327; 5,472,672; 5,527,681; 5,529,756; 5,545,531; 5,554,501; 5,561,071; 5,571,639; 5,593,839; 5,599,695; 5,624,711; 5,658,734; and 5,700,637.

Microarray technology allows for the measurement of the steady-state mRNA level of thousands of genes simultaneously thereby presenting a powerful tool for identifying effects such as the onset, arrest, or modulation of uncontrolled cell proliferation. Two microarray technologies are currently in wide use. The first are cDNA arrays and the second are oligonucleotide arrays. Although differences exist in the construction of these chips, essentially all downstream data analysis and output are the same. The product of these analyses are typically measurements of the intensity of the signal received from a labeled probe used to detect a cDNA sequence from the sample that hybridizes to a nucleic acid sequence at a known location on the microarray. Typically, the intensity of the signal is proportional to the quantity of cDNA, and thus mRNA, expressed in the sample cells. A large number of such techniques are available and useful. Preferred methods for determining gene expression can be found in U.S. Pat. Nos. 6,004,755; 6,218,114; 6,218,122; and 6,271,002.

Analysis of expression levels is conducted by comparing signal intensities. This is best done by generating a ratio matrix of the expression intensities of genes in a test sample versus those in a control sample. For instance, the gene expression intensities from a diseased tissue can be compared with the expression intensities generated from normal tissue of the same type (e.g., diseased breast tissue sample vs. normal breast tissue sample). A ratio of these expression intensities indicates the fold-change in gene expression between the test and control samples.

Gene expression profiles can also be displayed in a number of ways. The most common method is to arrange raw fluorescence intensities or ratio matrix into a graphical Dendogram where columns indicate test samples and rows indicate genes. The data are arranged so genes that have similar expression profiles are proximal to each other. The expression ratio for each gene is visualized as a color. For example, a ratio less than one (indicating down-regulation) may appear in the blue portion of the spectrum while a ratio greater than one (indicating up-regulation) may appear as a color in the red portion of the spectrum. Commercially available computer software programs are available to display such data including GeneSpring from Agilent Technologies and Partek Discover™ and Partek Infer™ software from Partek®.

Modulated genes used in the methods of the invention are described in the Examples. Differentially expressed genes are either up- or down-regulated in patients with a relapse of breast cancer relative to those without a relapse. Up regulation and down regulation are relative terms meaning that a detectable difference (beyond the contribution of noise in the system used to measure it) is found in the amount of expression of the genes relative to some baseline. In this case, the baseline is the measured gene expression of a non-relapsing patient. The genes of interest in the diseased cells (from the relapsing patients) are then either up- or down-regulated relative to the baseline level using the same measurement method. Diseased, in this context, refers to an alteration of the state of a body that interrupts or disturbs, or has the potential to disturb, proper performance of bodily functions as occurs with the uncontrolled proliferation of cells. Someone is diagnosed with a disease when some aspect of that person's genotype or phenotype is consistent with the presence of the disease. However, the act of conducting a diagnosis or prognosis includes the determination of disease/status issues such as determining the likelihood of relapse and therapy monitoring. In therapy monitoring, clinical judgments are made regarding the effect of a given course of therapy by comparing the expression of genes over time to determine whether the gene expression profiles have changed or are changing to patterns more consistent with normal tissue.

Preferably, levels of up- and down-regulation are distinguished based on fold changes of the intensity measurements of hybridized microarray probes. A 2.0 fold difference is preferred for making such distinctions (or a p-value less than 0.05). That is, before a gene is said to be differentially expressed in diseased/relapsing versus normal/non-relapsing cells, the diseased cell is found to yield at least 2 times more, or 2 times less intensity than the normal cells. The greater the fold difference, the more preferred is use of the gene as a diagnostic or prognostic tool. Genes selected for the gene expression profiles of the instant invention have expression levels that result in the generation of a signal that is distinguishable from those of the normal or non-modulated genes by an amount that exceeds background using clinical laboratory instrumentation.

Statistical values can be used to confidently distinguish modulated from non-modulated genes and noise. Statistical tests find the genes most significantly different between diverse groups of samples. The Student's T-test is an example of a robust statistical test that can be used to find significant differences between two groups. The lower the p-value, the more compelling the evidence that the gene is showing a difference between the different groups. Nevertheless, since microarrays measure more than one gene at a time, tens of thousands of statistical tests may be performed at one time. Because of this, one is unlikely to see small p-values just by chance and adjustments for this using a Sidak correction as well as a randomization/permutation experiment can be made. A p-value less than 0.05 by the T-test is evidence that the gene is significantly different. More compelling evidence is a p-value less then 0.05 after the Sidak correction is factored in. For a large number of samples in each group, a p-value less than 0.05 after the randomization/permutation test is the most compelling evidence of a significant difference.

Another parameter that can be used to select genes that generate a signal that is greater than that of the non-modulated gene or noise is the use of a measurement of absolute signal difference. Preferably, the signal generated by the modulated gene expression is at least 20% different than those of the normal or non-modulated gene (on an absolute basis). It is even more preferred that such genes produce expression patterns that are at least 30% different than those of normal or non-modulated genes.

Genes can be grouped so that information obtained about the set of genes in the group provides a sound basis for making a clinically relevant judgment such as a diagnosis, prognosis, or treatment choice. These sets of genes make up the portfolios of the invention. In this case, the judgments supported by the portfolios involve breast cancer and its chance of recurrence. As with most diagnostic markers, it is often desirable to use the fewest number of markers sufficient to make a correct medical judgment. This prevents a delay in treatment pending further analysis as well inappropriate use of time and resources.

Preferably, portfolios are established such that the combination of genes in the portfolio exhibit improved sensitivity and specificity relative to individual genes or randomly selected combinations of genes. In the context of the instant invention, the sensitivity of the portfolio can be reflected in the fold differences exhibited by a gene's expression in the diseased state relative to the normal state. Specificity can be reflected in statistical measurements of the correlation of the signaling of gene expression with the condition of interest. For example, standard deviation can be a used as such a measurement. In considering a group of genes for inclusion in a portfolio, a small standard deviation in expression measurements correlates with greater specificity. Other measurements of variation such as correlation coefficients can also be used.

One method of establishing gene expression portfolios is through the use of optimization algorithms such as the mean variance algorithm widely used in establishing stock portfolios. This method is described in detail in US patent publication number 20030194734. Essentially, the method calls for the establishment of a set of inputs (stocks in financial applications, expression as measured by intensity here) that will optimize the return (e.g., signal that is generated) one receives for using it while minimizing the variability of the return. Many commercial software programs are available to conduct such operations. “Wagner Associates Mean-Variance Optimization Application,” referred to as “Wagner Software” throughout this specification, is preferred. This software uses functions from the “Wagner Associates Mean-Variance Optimization Library” to determine an efficient frontier and optimal portfolios in the Markowitz sense is preferred. Use of this type of software requires that microarray data be transformed so that it can be treated as an input in the way stock return and risk measurements are used when the software is used for its intended financial analysis purposes.

The process of selecting a portfolio can also include the application of heuristic rules. Preferably, such rules are formulated based on biology and an understanding of the technology used to produce clinical results. More preferably, they are applied to output from the optimization method. For example, the mean variance method of portfolio selection can be applied to microarray data for a number of genes differentially expressed in subjects with breast cancer. Output from the method would be an optimized set of genes that could include some genes that are expressed in peripheral blood as well as in diseased tissue. If samples used in the testing method are obtained from peripheral blood and certain genes differentially expressed in instances of breast cancer are differentially expressed in peripheral blood, then a heuristic rule can be applied in which a portfolio is selected from the efficient frontier excluding those that are differentially expressed in peripheral blood. Of course, the rule can be applied prior to the formation of the efficient frontier by, for example, applying the rule during data pre-selection.

Other heuristic rules can be applied that are not necessarily related to the biology in question. For example, one can apply a rule that only a prescribed percentage of the portfolio can be represented by a particular gene or group of genes. Commercially available software such as the Wagner Software readily accommodates these types of heuristics. This can be useful, for example, when factors other than accuracy and precision (e.g., anticipated licensing fees) have an impact on the desirability of including one or more genes.

One method of the invention involves comparing gene expression profiles for various genes (or portfolios) to ascribe prognoses. The gene expression profiles of each of the genes comprising the portfolio are fixed in a medium such as a computer readable medium. This can take a number of forms. For example, a table can be established into which the range of signals (e.g., intensity measurements) indicative of disease is input. Actual patient data can then be compared to the values in the table to determine whether the patient samples are normal or diseased. In a more sophisticated embodiment, patterns of the expression signals (e.g., fluorescent intensity) are recorded digitally or graphically.

The gene expression patterns from the gene portfolios used in conjunction with patient samples are then compared to the expression patterns. Pattern comparison software can then be used to determine whether the patient samples have a pattern indicative of recurrence of the disease. Of course, these comparisons can also be used to determine whether the patient is not likely to experience disease recurrence. The expression profiles of the samples are then compared to the portfolio of a control cell. If the sample expression patterns are consistent with the expression pattern for recurrence of a breast cancer then (in the absence of countervailing medical considerations) the patient is treated as one would treat a relapse patient. If the sample expression patterns are consistent with the expression pattern from the normal/control cell then the patient is diagnosed negative for breast cancer.

In this invention, the most preferred method for analyzing the gene expression pattern of a patient to determine prognosis of breast cancer is through the use of a Cox's hazard analysis program. Most preferably, the analysis is conducted using S-Plus software (commercially available from Insightful Corporation). Using such methods, a gene expression profile is compared to that of a profile that confidently represents relapse (i.e., expression levels for the combination of genes in the profile is indicative of relapse). The Cox's hazard model with the established threshold is used to compare the similarity of the two profiles (known relapse versus patient). and then determines whether the patient profile exceeds the threshold. If it does, then the patient is classified as one who will relapse and is accorded treatment such as adjuvant therapy. If the patient profile does not exceed the threshold then they are classified as a non-relapsing patient. Other analytical tools can also be used to answer the same question such as, linear discriminate analysis, logistic regression and neural network approaches.

Numerous other well-known methods of pattern recognition are available. The following references provide some examples: Weighted Voting: Golub et al. (1999); Support Vector Machines: Su et al. (2001); and Ramaswamy et al. (2001); K-nearest Neighbors: Ramaswamy (2001); and Correlation Coefficients: van't Veer et al. (2002).

The gene expression profiles of this invention can also be used in conjunction with other non-genetic diagnostic methods useful in cancer diagnosis, prognosis, or treatment monitoring. For example, in some circumstances it is beneficial to combine the diagnostic power of the gene expression based methods described above with data from conventional markers such as serum protein markers (e.g., Cancer Antigen 27.29 (“CA 27.29”)). A range of such markers exists including such analytes as CA 27.29. In one such method, blood is periodically taken from a treated patient and then subjected to an enzyme immunoassay for one of the serum markers described above. When the concentration of the marker suggests the return of tumors or failure of therapy, a sample source amenable to gene expression analysis is taken. Where a suspicious mass exists, a fine needle aspirate (FNA) is taken and gene expression profiles of cells taken from the mass are then analyzed as described above. Alternatively, tissue samples may be taken from areas adjacent to the tissue from which a tumor was previously removed. This approach can be particularly useful when other testing produces ambiguous results.

Articles of this invention include representations of the gene expression profiles useful for treating, diagnosing, prognosticating, and otherwise assessing diseases. These profile representations are reduced to a medium that can be automatically read by a machine such as computer readable media (magnetic, optical, and the like). The articles can also include instructions for assessing the gene expression profiles in such media. For example, the articles may comprise a CD ROM having computer instructions for comparing gene expression profiles of the portfolios of genes described above. The articles may also have gene expression profiles digitally recorded therein so that they may be compared with gene expression data from patient samples. Alternatively, the profiles can be recorded in different representational format. A graphical recordation is one such format. Clustering algorithms such as those incorporated in Partek Discover™ and Partek Infer™ software from Partek® mentioned above can best assist in the visualization of such data.

Different types of articles of manufacture according to the invention are media or formatted assays used to reveal gene expression profiles. These can comprise, for example, microarrays in which sequence complements or probes are affixed to a matrix to which the sequences indicative of the genes of interest combine creating a readable determinant of their presence. Alternatively, articles according to the invention can be fashioned into reagent kits for conducting hybridization, amplification, and signal generation indicative of the level of expression of the genes of interest for detecting breast cancer.

Kits made according to the invention include formatted assays for determining the gene expression profiles. These can include all or some of the materials needed to conduct the assays such as reagents and instructions.

SEQ ID NOs: 1-197 are summarized in Table 5. In each SEQ ID NO: 1-197, the marker is identified by a psid or Affymetrix Proset ID represents the gene encoding any variant, allele etc. corresponding to the given SEQ ID NO. The marker is also defined as the gene encoding mRNA recognized by the probe corresponding to the given psid.

The following examples are provided to illustrate but not limit the claimed invention. All references cited herein are hereby incorporated herein by reference. Genes analyzed according to this invention are typically related to full-length nucleic acid sequences that code for the production of a protein or peptide. One skilled in the art will recognize that identification of full-length sequences is not necessary from an analytical point of view. That is, portions of the sequences or ESTs can be selected according to well-known principles for which probes can be designed to assess gene expression for the corresponding gene.

### EXAMPLE 1

### Comparison of Expression Intensities of 21 Consecutively Expressed Housekeeping Genes Between the Bulk Tumor Data Set and the LCM-Procured Sample Data Set

In order to gain insights into the mechanisms trigged by estrogen in breast epithelia cells, we applied LCM technique to a set of 28 early stage primary breast tumors that consisted of 17 ER+ and 11 ER− tumors. We then analyzed their gene expression profiles using Affymetrix GeneChip Hu133A.

Breast tumors used in this study were selected from the Erasmus Medical Center tumor bank, Rotterdam, Netherlands. These samples were submitted to the laboratory for routine assessment of steroid hormone receptor status, and stored since in liquid nitrogen. The present study in which coded tumor tissues were used was performed according to the Code of Conduct of the Federation of Medical Scientific Societies in the Netherlands. The study was approved by the institutional Medical Ethical Committee of the Erasmus Medical Center. Patients were diagnosed as stage I and IIa between 1983 and 1994, with a median age of 49 years (range: 29-74 years). These breast tumors are mostly invasive ductal carcinoma (IDC) with a fraction of tumors having co-existing ductal carcinoma in situ (DCIS). Table 1 specifies clinical characteristics of these patients. 25 patients had breast-conserving surgery and 3 had mastectomy.

| Characteristic | # of patients (%) |
|---|---|
| Age (yr) | |
| <40 | 2 (7) |
| 40-44 | 5 (17) |
| 45-49 | 8 (27) |
| >=50 | 15 (50) |
| Tumor diameter (mm) | |
| <=20 | 11 (37) |
| >20 | 18 (60) |
| Histologic grade | |
| II (intermediate) | 5 (17) |
| III (poor) | 12 (40) |
| Lymph node metastasis | |
| Negative | 28 (100) |
| Surgery | |
| Breast conserving | 25 (89) |
| Mastectomy | 3 (11) |
| Chemotherapy | |
| No | 28 (100) |
| Hormonal | |
| No | 28 (100) |

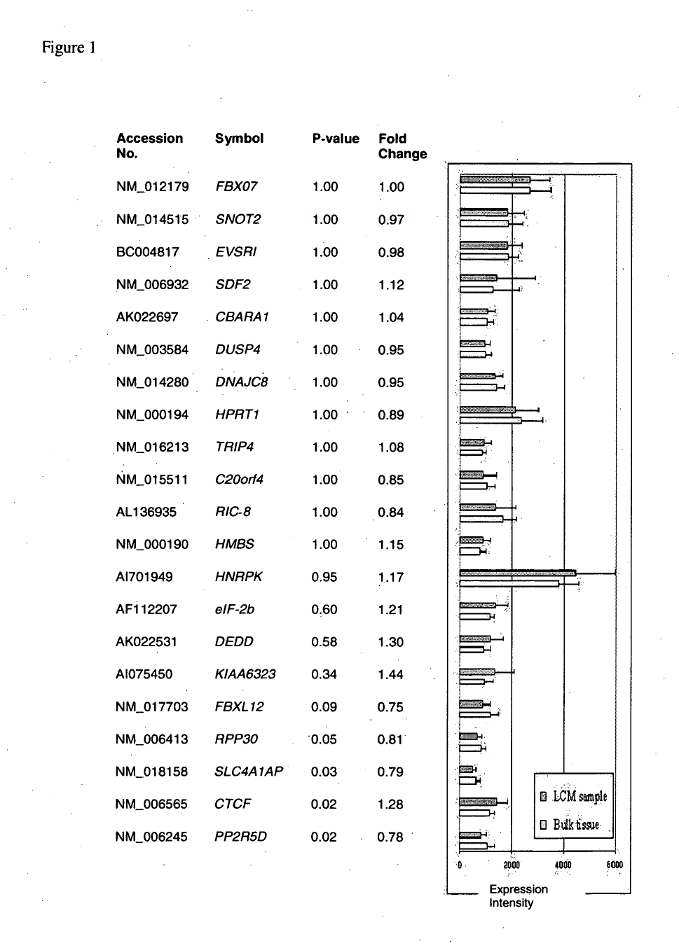

All patients had dissection of the axillary lymph nodes, following by radiotherapy if indicated. No neo-adjuvant treatment had been administered. Tumors were characterized as primary invasive breast carcinoma with size less than 5 cm in diameter (pT1 or pT2). No lymph node metastasis was found at the time of surgery. ER status was determined by ligand-binding assay or enzyme immunoassay as described. Foekens et al. (1989). To classify tumors as ER+ or ER− a cutoff of 10 fmol/mg cytosolic protein was used. To produce the gene expression profiles, an average of 1,000 tumor cells were procured from fresh-frozen sections of the tumor block. A T7-based RNA linear amplification was carried out to obtain sufficient amounts of biotin-labeled aRNA for microarray analysis. Kamme et al. (2004). Using TargetAmp RNA amplification kit (Epicenter, WI) with the biotin-labeling step being substituted with Affymetrix Enzo kit (Affymetrix, CA) in the second round of amplification, in average, 60 μg of aRNA was generated after two rounds of amplification, with a mean size distribution of approximately 2,000 nucleotides. The amplification power was roughly 10⁶-fold from the initial total RNA. Linear regression analysis of the gene expression data derived from the replicates of amplified RNA indicated an R² value of 0.96. The good fidelity and reproducibility of two rounds T7-based RNA linear amplification have been demonstrated in several reports (Luzzi et al. 2003; and Ma et al. 2003), and amplification on the 3′ end of transcripts does not have a major impact on the overall transcript profiles with Affymetrix GeneChip® because the probe sets on the array are designed using 3′ end sequences. Luzzi et al. (2003). Furthermore, the amplification method was shown to enhance sensitivity of identifying the differentially expressed genes as compared to the un-amplified method. Polacek et al. (2003). To ensure that the amplification method in our study accurately preserves mRNA abundance in LCM derived RNA samples, the expression levels of 21 constitutively expressed housekeeping genes were compared between the LCM-procured samples and the bulk tumor samples (FIG. 1). These 21 genes were selected based on a large collection of gene expression profiles from normal, benign, and tumor samples across different tissue types. There is no statistically significant difference between the expression levels of these 21 genes from the two-round amplified LCM samples and their corresponding bulk tumor samples. Therefore, our result agrees with the published studies that two rounds of T7-based RNA amplification accurately preserve the mRNA abundance in the RNA samples, and the combination of LCM and RNA amplification is a reliable approach for gene expression profiling.

To generate the bulk tumor gene expression data, total RNA samples were extracted using Trizol method (Invitrogen, CA). The targets were then biotin-labeled and hybridized to GeneChip Hu133A according to the manufacturer's manual (Affymetrix, CA). For the LCM-procured sample data set, tumor cells were procured using the PALM® Microlaser system and ZEISS Axiovert 135 (P.A.L.M. Microlaser Technologies, Germany) and an established protocol. Kamme et al. (2004). In brief, embedded frozen tumor specimens were cut as a series of 100 μm thick sections on a Cryocut 1800 Reichert-Jung cryotome (Cambridge Instruments, Germany) at a temperature between −17° C. to −25° C., and were mounted on PEN (polyethylene naphthalate) membrane slides (P.A.L.M. Microlaser Technologies, Germany). Tissue sections were immediately fixed in 100% cold ethanol.

For H&E staining, slides were sequentially dipped five times in a series of ethanol solutions with decreasing concentrations, 30 seconds in Harris hematoxylin solution (Sigma, St. Louis, Mo.), briefly washed with DI water, five times in Eosin Y (Sigma, St. Louis, Mo.), rinsed with 95% ethanol and 100% ethanol. Slides were ready for LCM procedure after 10 minutes of air drying. For each tumor sample, the first and the last tissue section were mounted on a glass slide and embedded in xylene after H&E staining, which served as the reference and the confirmation for diagnosis. Areas containing tumor cells were then independently isolated from the slides and stored in 100% ethanol. Total RNA from laser-captured cells was extracted with RNeasy buffers (Qiagen, Germany) and recovered using Zymo spin-column (Zymo Research, CA). The RNA samples were then amplified with TargetAmp™ kit with modifications as stated in the text. The final biotin-labeled aRNA product was hybridized to GeneChip Hu133A. For data analysis, the images from the scanned chips were processed using Microarray Analysis Suite 5.0 (Affymetrix Inc., CA). Image data from each microarray was individually scaled to an average intensity of 600. Quality control standards were as follows: RawQ less than 4, background less than 100, scaling factor less than 4, and percentage of “present” call was more than 35%. Blue and yellow bars represent expression levels in the bulk tumors and LCM samples, respectively. Error bars represent the standard deviation across 28 experiments in each data set. P-value was obtained using the T-test. P-value less than 0.01 were considered significantly different between the two data sets. The results are depicted in FIG. 1.

### EXAMPLE 2

### Unsupervised Two-Dimensional Hierarchical Clustering Analysis of the Global Gene Expression Data Using Gene Spring Software

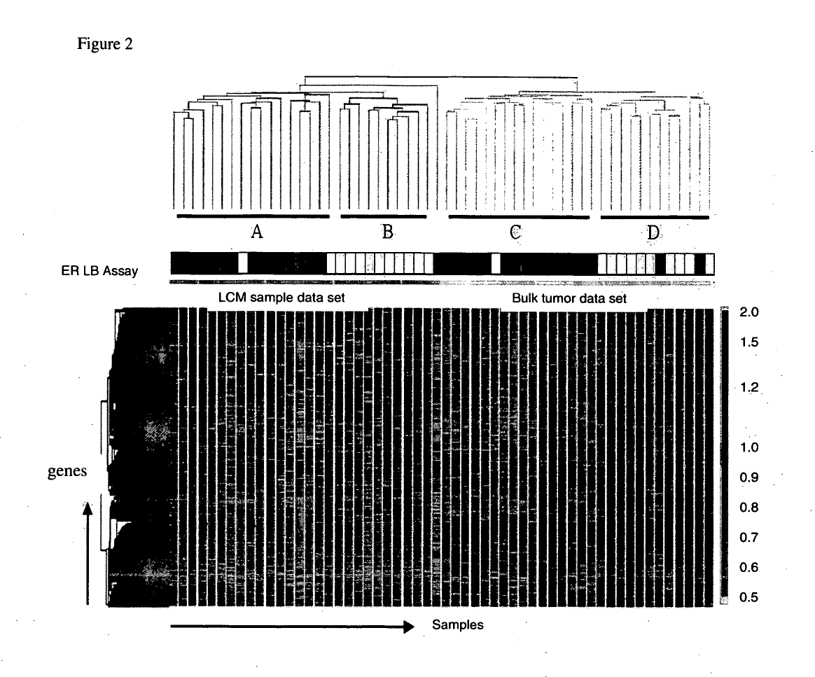

Gene expression intensities of approximately 23,000 probe sets on Affymetrix UI 33A chip were first normalized using a quantile normalization method, then filtered using “present” call determined by Affymetrix MAS 5.0 software. An unsupervised two-dimensional hierarchical clustering algorithm was applied to the microarray data in order to group genes on the basis of similarities in the expression patterns and to cluster samples on the basis of similarities in the global gene expression profiles. As shown in FIG. 2, 56 samples (28 LCM+28 bulk tissue) were clustered into two major groups according to the source of RNA extraction: LCM-procured tumor cells and mixed cell population from bulk tumors. In each group, the samples were further clustered into two sub-groups (group A and B in LCM samples, group C and D in bulk tissue samples). As we investigated the possible association of clinical parameters to these sub-groups, ER status has the most significant correlation with the classification. In the LCM data set, of 17 tumors diagnosed as ER+, 15 were classified into the same sub-group (group A), and one formed its own subgroup and one being classified into ER− group. 10 out of 11 ER− tumors were classified into sub-group B, with the estimated P-value of X² test being 0.0006. One ER− tumor was clustered with ER+ tumors. As for the bulk tumor data set, the same 15 ER+ samples were classified in the correct category (sub-group C), and the same single ER− sample was clustered with the ER+ group. The two ER+ tumors that were classified into ER− sub-group had very low expression of estrogen receptor in the chip data, while the one ER− tumor that was classified with ER+ subgroup had high expression of ER on the chip. The discrepancy between the routine assessed ER status and the gene expression data may be due to the heterogeneity of tumors or the post-transcriptional regulation of ER expression in these tumors.

### EXAMPLE 3

### Pathway Analyses of Differentially Expressed Genes Between ER+ Subgroup and ER− Subgroup

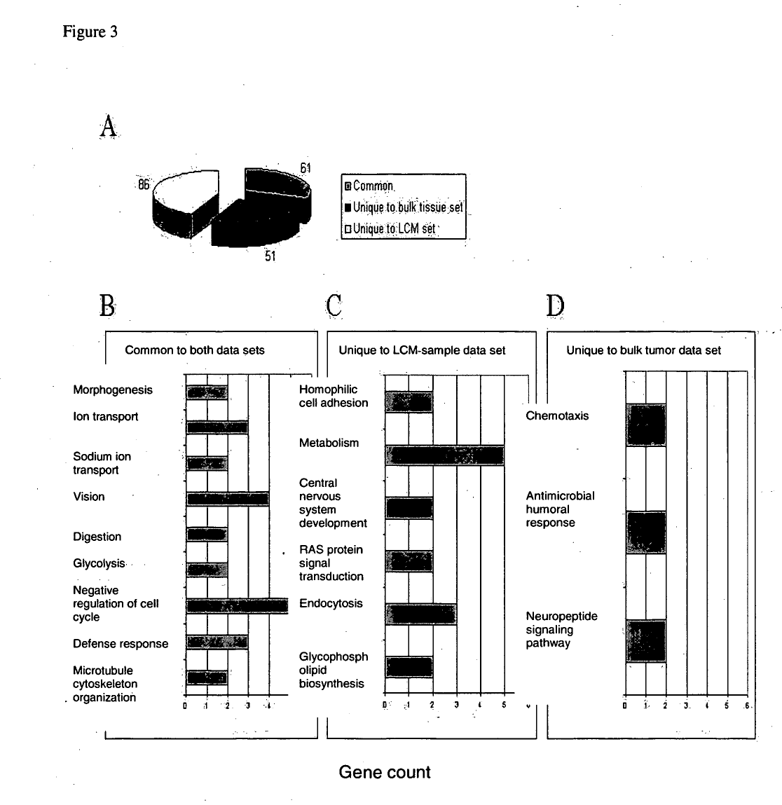

To identify genes associated with ER status and its related pathways, we carried out T-test between the ER+ subgroup and the ER− subgroup in each of the two data sets. Using the Bonferroni corrected P-value <0.05 as a cutoff, 175 probe sets representing 146 unique genes were found in the LCM-procured sample data set and 130 probe sets representing 112 unique genes were identified in the bulk tumor data set. By comparing these two gene lists, 61 genes were found to be common, 85 genes were unique to the LCM-procured samples, and 51 genes were only present in the bulk tumor samples (FIG. 3A; Tables 2, 3 and 4). Of the 61 common genes, 36 were relatively over-expressed and 25 were down-regulated in the ER+ subgroup (Table 2). Estrogen receptor together with other genes known to be associated with ER activation, such as trefoil factors 1 & 3, GATA3, X-box binding protein 1 (XBP1), and keratin 18 were among the up-regulated genes. Sotiriou et al. (2003); Gruvberger et al. (2001); and Sun et al. (2005). On the other hand, P-cadherin (CDH3), GABRP, and secreted frizzled-related protein 1 (SFRP1) were present in the down-regulated gene list.

TABLE 2
Common genes in both LCM sample data set and tumor mass data set
with altered expression between ER+ and ER− tumors

| SEQ ID NO | Affymetrix Proset ID | p-value | Relative expression* |
|---|---|---|---|
| 23 | 200670_at | 1.03E−07 | + |
| 33 | 200747_s_at | 3.24E−07 | + |
| 5 | 200811_at | 7.35E−10 | + |
| 40 | 201030_x_at | 5.11E−07 | − |
| 29 | 201596_x_at | 1.92E−07 | + |
| 24 | 201795_at | 1.12E−07 | − |
| 61 | 202035_s_at | 3.13E−06 | − |
| 48 | 202342_s_at | 1.14E−06 | − |
| 58 | 202345_s_at | 2.83E−06 | − |
| 53 | 202554_s_at | 1.67E−06 | + |
| 46 | 202908_at | 9.59E−07 | + |
| 60 | 203256_at | 3.03E−06 | − |
| 34 | 203263_s_at | 3.49E−07 | − |
| 49 | 203453_at | 1.17E−06 | + |
| 13 | 203712_at | 3.09E−08 | − |
| 35 | 203909_at | 3.51E−07 | − |
| 3 | 203928_x_at | 2.45E−10 | + |
| 11 | 204304_s_at | 9.30E−09 | − |
| 8 | 204508_s_at | 4.40E−09 | + |
| 6 | 204623_at | 1.11E−09 | + |
| 43 | 204872_at | 7.34E−07 | − |
| 54 | 204915_s_at | 2.58E−06 | − |
| 14 | 205009_at | 4.34E−08 | + |
| 19 | 205044_at | 7.53E−08 | − |
| 7 | 205225_at | 1.99E−09 | + |
| 59 | 205376_at | 3.00E−06 | + |
| 27 | 205548_s_at | 1.85E−07 | + |
| 51 | 205862_at | 1.24E−06 | + |
| 30 | 206392_s_at | 2.02E−07 | − |
| 15 | 206755_at | 4.71E−08 | + |
| 57 | 206838_at | 2.79E−06 | − |
| 32 | 207828_s_at | 3.22E−07 | − |
| 2 | 208682_s_at | 5.33E−11 | + |
| 39 | 209191_at | 4.94E−07 | − |
| 12 | 209460_at | 9.44E−09 | + |
| 22 | 209602_s_at | 9.40E−08 | + |
| 17 | 209696_at | 5.00E−08 | + |
| 26 | 209791_at | 1.51E−07 | − |
| 9 | 210347_s_at | 4.60E−09 | − |
| 41 | 211712_s_at | 5.15E−07 | + |
| 37 | 212441_at | 4.65E−07 | + |
| 1 | 212494_at | 4.31E−11 | + |
| 55 | 212496_s_at | 2.66E−06 | + |
| 21 | 212638_s_at | 8.19E−08 | + |
| 18 | 212692_s_at | 6.49E−08 | + |
| 38 | 212744_at | 4.72E−07 | + |
| 25 | 212956_at | 1.46E−07 | + |
| 47 | 212985_at | 1.12E−06 | + |
| 42 | 213923_at | 5.43E−07 | − |
| 10 | 214053_at | 7.33E−09 | + |
| 16 | 214440_at | 4.94E−08 | + |
| 20 | 214745_at | 8.03E−08 | − |
| 4 | 216092_s_at | 2.94E−10 | + |
| 52 | 218211_s_at | 1.30E−06 | + |
| 45 | 219197_s_at | 7.64E−07 | + |
| 56 | 220016_at | 2.76E−06 | + |
| 31 | 220230_s_at | 2.98E−07 | − |
| 28 | 220540_at | 1.85E−07 | − |
| 50 | 221203_s_at | 1.20E−06 | − |
| 36 | 221920_s_at | 3.63E−07 | − |
| 44 | 222125_s_at | 7.43E−07 | + |

TABLE 3
Genes unique in bulk tumor data set with altered expression between
ER+ and ER− tumors

| SEQ ID NO | Affymetrix Proset ID | p-value | Relative expression* |
|---|---|---|---|
| 92 | 200719_at | 7.48E−07 | + |
| 66 | 200804_at | 2.13E−08 | + |
| 65 | 201037_at | 1.46E−08 | − |
| 93 | 201754_at | 8.40E−07 | + |
| 100 | 202089_s_at | 1.32E−06 | + |
| 67 | 202897_at | 2.52E−08 | − |
| 62 | 202982_s_at | 2.58E−09 | + |
| 111 | 203287_at | 2.95E−06 | − |
| 110 | 203773_x_at | 2.83E−06 | + |
| 83 | 204284_at | 4.04E−07 | + |
| 73 | 204540_at | 1.60E−07 | + |
| 97 | 204567_s_at | 1.01E−06 | + |
| 90 | 204667_at | 6.42E−07 | + |
| 64 | 204822_at | 1.03E−08 | − |
| 105 | 205081_at | 1.95E−06 | + |
| 63 | 205186_at | 6.07E−09 | + |
| 112 | 206249_at | 2.99E−06 | − |
| 88 | 207571_x_at | 6.14E−07 | − |
| 80 | 208788_at | 3.37E−07 | + |
| 77 | 208873_s_at | 2.82E−07 | + |
| 72 | 209114_at | 1.54E−07 | + |
| 69 | 209122_at | 2.87E−08 | − |
| 103 | 209324_s_at | 1.88E−06 | − |
| 96 | 209870_s_at | 9.47E−07 | − |
| 98 | 210397_at | 1.23E−06 | − |
| 75 | 210845_s_at | 2.22E−07 | − |
| 85 | 211063_s_at | 5.32E−07 | − |
| 74 | 211967_at | 2.12E−07 | − |
| 70 | 212276_at | 4.99E−08 | − |
| 81 | 212501_at | 3.47E−07 | − |
| 76 | 213634_s_at | 2.33E−07 | + |
| 94 | 213651_at | 8.46E−07 | + |
| 101 | 214431_at | 1.37E−06 | − |
| 71 | 215304_at | 8.52E−08 | + |
| 107 | 215329_s_at | 2.27E−06 | + |
| 89 | 216988_s_at | 6.25E−07 | + |
| 108 | 217979_at | 2.28E−06 | + |
| 95 | 218104_at | 8.55E−07 | − |
| 87 | 218195_at | 5.72E−07 | + |
| 106 | 218239_s_at | 2.22E−06 | − |
| 104 | 218532_s_at | 1.92E−06 | + |
| 78 | 218534_s_at | 3.10E−07 | + |
| 91 | 218854_at | 6.51E−07 | − |
| 109 | 218966_at | 2.40E−06 | + |
| 99 | 219615_s_at | 1.25E−06 | − |
| 84 | 219918_s_at | 4.21E−07 | − |
| 79 | 220425_x_at | 3.22E−07 | − |
| 86 | 221834_at | 5.37E−07 | + |
| 68 | 221934_s_at | 2.72E−08 | + |
| 82 | 51158_at | 3.56E−07 | + |
| 102 | 60471_at | 1.46E−06 | − |

TABLE 4
Genes unique in LCM sample data set with altered expression between
ER+ and ER− tumors

| SEQ ID NO | Affymetrix Proset ID | p-value | Relative expression* |
|---|---|---|---|
| 193 | 200790_at | 2.38E−06 | − |
| 113 | 200824_at | 1.08E−09 | − |
| 182 | 201012_at | 2.06E−06 | − |
| 176 | 201215_at | 1.92E−06 | + |
| 133 | 201300_s_at | 2.77E−07 | − |
| 164 | 201407_s_at | 1.39E−06 | − |
| 137 | 201564_s_at | 3.84E−07 | − |
| 150 | 201636_at | 9.98E−07 | − |
| 128 | 201833_at | 2.36E−07 | − |
| 152 | 201915_at | 1.02E−06 | − |
| 120 | 201980_s_at | 1.29E−07 | − |
| 158 | 202121_s_at | 1.22E−06 | + |
| 192 | 202146_at | 2.36E−06 | − |
| 142 | 202207_at | 6.93E−07 | − |
| 140 | 202320_at | 5.21E−07 | + |
| 189 | 202772_at | 2.26E−06 | + |
| 117 | 203384_s_at | 3.26E−08 | + |
| 121 | 203682_s_at | 1.30E−07 | + |
| 115 | 203702_s_at | 2.17E−08 | − |
| 149 | 204688_at | 9.46E−07 | − |
| 125 | 204751_x_at | 1.97E−07 | − |
| 183 | 204785_x_at | 2.12E−06 | − |
| 126 | 204881_s_at | 2.07E−07 | + |
| 159 | 205109_s_at | 1.27E−06 | − |
| 127 | 205300_s_at | 2.16E−07 | + |
| 122 | 205363_at | 1.31E−07 | − |
| 186 | 205429_s_at | 2.21E−06 | − |
| 123 | 205471_s_at | 1.65E−07 | + |
| 195 | 205996_s_at | 2.53E−06 | − |
| 167 | 206364_at | 1.53E−06 | − |
| 170 | 206565_x_at | 1.61E−06 | + |
| 155 | 208103_s_at | 1.15E−06 | − |
| 114 | 208358_s_at | 1.05E−08 | − |
| 151 | 209025_s_at | 1.01E−06 | − |
| 178 | 209170_s_at | 1.97E−06 | − |
| 175 | 209173_at | 1.90E−06 | + |
| 143 | 209396_s_at | 6.97E−07 | − |
| 147 | 209494_s_at | 8.55E−07 | + |
| 130 | 209531_at | 2.54E−07 | + |
| 160 | 209631_s_at | 1.30E−06 | − |
| 179 | 209745_at | 2.00E−06 | + |
| 169 | 210319_x_at | 1.59E−06 | + |
| 163 | 210466_s_at | 1.38E−06 | − |
| 194 | 210648_x_at | 2.43E−06 | − |
| 188 | 210687_at | 2.23E−06 | + |
| 168 | 210886_x_at | 1.54E−06 | + |
| 154 | 210942_s_at | 1.07E−06 | − |
| 180 | 211110_s_at | 2.03E−06 | + |
| 146 | 212314_at | 8.36E−07 | − |
| 190 | 212442_s_at | 2.28E−06 | + |
| 196 | 212462_at | 2.65E−06 | + |
| 181 | 212508_at | 2.06E−06 | + |
| 174 | 212759_s_at | 1.88E−06 | − |
| 156 | 212780_at | 1.17E−06 | − |
| 161 | 212846_at | 1.35E−06 | − |
| 136 | 213260_at | 3.42E−07 | − |
| 162 | 213419_at | 1.37E−06 | + |
| 197 | 214806_at | 2.87E−06 | − |
| 173 | 215723_s_at | 1.85E−06 | − |
| 187 | 217028_at | 2.22E−06 | − |
| 138 | 217823_s_at | 4.37E−07 | − |
| 132 | 217838_s_at | 2.60E−07 | + |
| 131 | 217929_s_at | 2.55E−07 | + |
| 118 | 218236_s_at | 5.30E−08 | − |
| 184 | 218440_at | 2.13E−06 | − |
| 119 | 218483_s_at | 1.17E−07 | + |
| 172 | 218489_s_at | 1.83E−06 | + |
| 165 | 218618_s_at | 1.44E−06 | − |
| 124 | 218931_at | 1.83E−07 | + |
| 157 | 219010_at | 1.20E−06 | − |
| 129 | 219100_at | 2.40E−07 | + |
| 134 | 219212_at | 2.87E−07 | − |
| 177 | 219562_at | 1.92E−06 | − |
| 145 | 219686_at | 8.16E−07 | + |
| 148 | 219806_s_at | 9.21E−07 | − |
| 185 | 219861_at | 2.19E−06 | + |
| 153 | 219889_at | 1.06E−06 | + |
| 139 | 220173_at | 4.62E−07 | + |
| 191 | 220432_s_at | 2.34E−06 | − |
| 166 | 220533_at | 1.51E−06 | − |
| 141 | 220658_s_at | 5.50E−07 | − |
| 171 | 221562_s_at | 1.67E−06 | + |
| 135 | 221641_s_at | 3.40E−07 | − |
| 144 | 222011_s_at | 7.79E−07 | − |
| 116 | 52940_at | 2.90E−08 | + |

Using Gene Ontology annotation, distinctive pathways were identified with P-value <0.05 for the three gene lists (FIGS. 3B, 3C, and 3D). For the 61 genes, the most significant pathway turned out to be the microtubule cytoskeleton organization pathway, followed by defense response, negative regulation of cell proliferation, glycolysis, digestion, vision, sodium ion-transport, ion-transport, and morphogenesis pathways. The negative regulation of cell proliferation pathway, in which retinoic acid receptor responder (tazarotene induced) 1 and BTG family member 3 genes were found down-regulated in ER+ tumors, has the most genes involved from the common gene list. An interesting discovery is the up-regulation in ER+ breast tumors of the microtubule-associated protein tau (MAPT) in the microtubule cytoskeleton organization and biogenesis pathway. This gene is differentially expressed in the nervous system (Binder et al. 1985) and its mutations result in several neurodegenerative disorders. Spillantini et al. (1998). Although its suppression in primate brains was reported in correlation with ingestion of phytoestrogen isoflavones (Kim et al. 2001), its up-regulation associated with ER status in breast tumor cells has not been shown before.

The significant pathways identified in the LCM sample unique gene list are the following: glycosphingolipid biosynthesis, endocytosis, RAS protein signal transduction, central nervous system development, metabolism, and homophilic cell adhesion. UDP-glucose ceramide glucosyltransferase and UDP glycosyltransferase 8 are involved in glycosphingolipid biosynthesis such as ceramide, which functions as a second messenger to signaling cascades that promote differentiation, senescence, proliferation, and apoptosis. Simstein et al. (2003). Although the mechanism underlying interactions within the ER pathway is unknown, ceramide generation was associated with tamoxifen-induced apoptosis (Mandlekar et al. 2001), and possibly interrupts estrogen's anti-apoptotic signaling pathways via the extracellular signal-regulated kinases (ERKs). Chen et al. (2005). Moreover, another identified pathway, endocytosis, has been associated with cell adhesion and migration in breast cancer via the Eph/Ephrin signaling pathway, which cross-activates the JAT-STAT pathway. Fox et al. 2004; and Poliakov et al. (2004). Members of the RAS oncogene family (RAB17 and RAB26) as well as genes involved in RAS protein signal transduction (SOS1 and PLD1) were identified. Hyperactive Ras can promote breast cancer growth and development, and affects upstream of the ERK/AKT signaling pathway. Eckert et al. (2004). It was demonstrated in MCF7 cells that Ras activity was required for nuclear export and degradation of p27 in response to estradiol and mediated a novel nongenomic pathway in promoting survival of breast cancer cells in culture. Fernando et al. (2004).

Transforming growth factor β (TGF-β) has been demonstrated to have both tumor suppression and stimulating effects during early and late stages of tumorigenesis. Akhurst et al. (2001). The cross talk between TGF-β signaling and estrogen signaling at DNA-dependent or -independent manners has been documented. Matsuda et al. (2001); Wu et al. (2003); and Ammanamanchi et al. (2004). A few genes that have implied action on TGF-β signaling were identified in the common and LCM unique gene lists. WW domain-containing protein 1 (WWP1), which is an E3 ubiquitin ligase expressed in epithelium was found to inhibit TGF-β signaling through inducing ubiquitination and degradation of the TGF-β type I receptor. Malbert-Colas et al. (2003); and Komuro et al. (2004). Sotiriou et al. (2003) also found this gene in their ER status associated gene list, although its interaction with the ER pathway is still unknown. DACH1 was shown to inhibit TGF-β induced apoptosis in breast cancer cell lines through binding Smad4, which is a transcription corepressor for ER-α by interacting with the AF1 domain of ER-α. Wu et al. (2003). FOXC1, a regulator of DACH1 (Tamimi et al. 2004), was also present in the LCM sample unique gene list. Up-regulation of WWP1 and DACH1 suggested that TGF-β signaling was suppressed in ER+ tumors. Further in the LCM unique gene list, there are genes involved in functions that have been related to the ER pathway, such as DNA-depended transcription regulation, cell surface receptor linked signal transduction, cell adhesion/motility, metabolic enzymes and apoptosis. Among them, some genes are known to interact with ER, such as HDAC2, ANXA1, and CCNB1. Additional investigation of the potential roles of these genes and their relations with ER may provide insights into estrogen signaling and the inter-relationships between these pathways.

On the other hand, for the genes that are unique to bulk tumor samples, pathways involved in chemotaxis and antimicrobial humoral response were ranked high. Cysteine-rich protein 1 (CRIP1) is produced in human peripheral blood mononuclear cells and is associated with host defense. Khoo et al. (1997). Ladinin 1 (LAD1) is a basement-membrane protein that may contribute to the stability of the association of the epithelial layers with the underlying mesenchyme. Marinkovich et al. (1996).

Although the foregoing invention has been described in some detail by way of illustration and example for purposes of clarity of understanding, the descriptions and examples should not be construed as limiting the scope of the invention.

TABLE 5
Sequence identifications

| | | | Accession | |
|---|---|---|---|---|
| SEQ ID NO | psid | Name | No | Description |
| 1 | 212494_at | TENC1 | AB028998 | Tensin like C1 domain containing phosphatase |
| 2 | 208682_s_at | MAGED2 | AF126181 | Melanoma antigen, family D, 2 |
| 3 | 203928_x_at | MAPT | AI870749 | Microtubule-associated protein tau |
| 4 | 216092_s_at | SLC7A8 | AL365347 | Solute carrier family 7 (cationic amino acid transporter, y+ system), mem 8 |
| 5 | 200811_at | CIRBP | NM_001280 | Cold inducible RNA binding protein |
| 6 | 204623_at | TFF3 | NM_003226 | Trefoil factor 3 (intestinal) |
| 7 | 205225_at | ESR1 | NM_000125 | Estrogen receptor 1 |
| 8 | 204508_s_at | CA12 | BC001012 | Carbonic anhydrase XII |
| 9 | 210347_s_at | BCL11A | AF080216 | B-cell CLL/lymphoma 11A (zinc finger protein) |
| 10 | 214053_at | ERBB4 | AW772192 | V-erb-a erythroblastic leukemia viral oncogene homolog 4 (avian) |
| 11 | 204304_s_at | PROM1 | NM_006017 | Prominin 1 |
| 12 | 209460_at | ABAT | AF237813 | 4-aminobutyrate aminotransferase |
| 13 | 203712_at | KIAA0020 | NM_014878 | KIAA0020 |
| 14 | 205009_at | TFF1 | NM_003225 | Trefoil factor 1 (breast cancer, estrogen-inducible sequence expressed in) |
| 15 | 206755_at | CYP2B6 | NM_000767 | Cytochrome P450, family 2, subfamily B, polypeptide 6 |
| 16 | 214440_at | NAT1 | NM_000662 | N-acetyltransferase 1 (arylamine N-acetyltransferase) |
| 17 | 209696_at | FBP1 | D26054 | Fructose-1,6-bisphosphatase 1 |
| 18 | 212692_s_at | LRBA | W60686 | LPS-responsive vesicle trafficking, beach and anchor containing |
| 19 | 205044_at | GABRP | NM_014211 | gamma-aminobutyric acid (GABA) A receptor, π |
| 20 | 214745_at | PLCL3 | AW665865 | phospholipase C-like 3 |
| 21 | 212638_s_at | WWP1 | BF131791 | WW domain containing E3 ubiquitin protein ligase 1 |
| 22 | 209602_s_at | GATA3 | AI796169 | GATA binding protein 3 |
| 23 | 200670_at | XBP1 | NM_005080 | X-box binding protein 1 |
| 24 | 201795_at | LBR | NM_002296 | Lamin B receptor |
| 25 | 212956_at | KIAA0882 | AI348094 | KIAA0882 protein |
| 26 | 209791_at | PADI2 | AL049569 | Peptidyl arginine deiminase, type II |
| 27 | 205548_s_at | KCNK15 | NM_022358 | Potassium channel, subfamily K, member 15 |
| 28 | 220540_at | BTG3 | NM_006806 | BTG family, member 3 |
| 29 | 201596_x_at | KRT18 | NM_000224 | Keratin 18 |
| 30 | 206392_s_at | RARRES1 | NM_002888 | Retinoic acid receptor responder (tazarotene induced) 1 |
| 31 | 220230_s_at | CYB5R2 | NM_016229 | Cytochrome b5 reductase b5R.2 |
| 32 | 207828_s_at | CENPF | NM_005196 | Centromere protein F, 350/400ka (mitosin) |
| 33 | 200747_s_at | NUMA1 | NM_006185 | Nuclear mitotic apparatus protein 1 |
| 34 | 203263_s_at | ARHGEF9 | AI625739 | Cdc42 guanine nucleotide exchange factor (GEF) 9 |
| 35 | 203909_at | SLC9A6 | NM_006359 | Solute carrier family 9 (sodium/hydrogen exchanger), isoform 6 |
| 36 | 221920_s_at | MSCP | BE677761 | Mitochondrial solute carrier protein |
| 37 | 212441_at | KIAA0232 | D86985 | KIAA0232 gene product |
| 38 | 212744_at | BBS4 | AI813772 | Bardet-Biedl syndrome 4 |
| 39 | 209191_at | MGC4083 | BC002654 | Tubulin β MGC4083 |
| 40 | 201030_x_at | LDHB | NM_002300 | Lactate dehydrogenase B |
| 41 | 211712_s_at | ANXA9 | BC005830 | Annexin A9 |
| 42 | 213923_at | RAP2B | AW005535 | Member of RAS oncogene family |
| 43 | 204872_at | TLE4 | NM_007005 | Transducin-like enhancer of split 4 (E(sp1) homolog, Drosophila) |
| 44 | 222125_s_at | PH-4 | BC000580 | Hypoxia-inducible factor prolyl 4-hydroxylase |
| 45 | 219197_s_at | SCUBE2 | NM_020974 | Signal peptide, CUB domain, EGF-like-2 |
| 46 | 202908_at | WFS1 | NM_006005 | Wolfram syndrome 1 (wolframin) |
| 47 | 212985_at | CCNB1 | BF115739 | Cyclin B1 |
| 48 | 202342_s_at | TRIM2 | NM_015271 | Tripartite motif-containing 2 |
| 49 | 203453_at | SCNN1A | NM_001038 | Sodium channel, nonvoltage-gated 1 α |
| 50 | 221203_s_at | YEATS2 | NM_018023 | YEATS domain containing 2 |
| 51 | 205862_at | GREB1 | NM_014668 | GREB1 protein |
| 52 | 218211_s_at | MLPH | NM_024101 | Melanophilin |
| 53 | 202554_s_at | GSTM3 | AL527430 | Glutathione S-transferase M3 (brain) |
| 54 | 204915_s_at | SOX11 | AB028641 | SRY (sex-determining region Y)-box 11 |
| 55 | 212496_s_at | JMJD2B | AW237172 | Jumonji domain containing 2B |
| 56 | 220016_at | MGC5395 | NM_024060 | Hypothetical protein MGC5395 |
| 57 | 206838_at | TBX19 | NM_005149 | T-box 19 |
| 58 | 202345_s_at | FABP5 | NM_001444 | Fatty acid binding protein 5 (psoriasis-associated) |
| 59 | 205376_at | INPP4B | NM_003866 | Inositol polyphosphate-4-phosphatase, type II, 105 kDa |
| 60 | 203256_at | CDH3 | NM_001793 | Cadherin 3, type 1, P-cadherin (placental) |
| 61 | 202035_s_at | SFRP1 | AF017987 | Secreted frizzled-related protein 1 |
| 62 | 202982_s_at | ZAP128 | NM_006821 | Peroxisomal long-chain acyl-coA thioesterase |
| 63 | 205186_at | DNALI1 | NM_003462 | Dynein, axonemal, light intermediate polypeptide 1 |
| 64 | 204822_at | TTK | NM_003318 | TTK protein kinase |
| 65 | 201037_at | PFKP | NM_002627 | Phosphofructokinase, platelet |
| 66 | 200804_at | TEGT | NM_003217 | Testis enhanced gene transcript (BAX inhibitor 1) |
| 67 | 202897_at | PTPNS1 | AB023430 | Protein tyrosine phosphatase, non-receptor type substrate 1 |
| 68 | 221934_s_at | FLJ10496 | BF941492 | Hypothetical protein FLJ10496 |
| 69 | 209122_at | ADFP | BC005127 | Adipose-differentiation related protein |
| 70 | 212276_at | LPIN1 | D80010 | Lipin-1 |
| 71 | 215304_at | NA | U79293 | Clone 23948 mRNA sequence |
| 72 | 209114_at | TSPAN-1 | AF133425 | Tetraspan 1 |
| 73 | 204540_at | EEF1A2 | NM_001958 | Eukaryotic translation elongation factor 1 α2 |
| 74 | 211967_at | PORIMIN | BG538627 | Pro-oncosis receptor inducing membrane injury gene |
| 75 | 210845_s_at | PLAUR | U08839 | Plasminogen activator, urokinase receptor |
| 76 | 213634_s_at | CELSR1 | AL031588 | Cadherin, EGF LAG seven-pass G-type receptor 1 (flamingo homolog, Drosophila) |
| 77 | 208873_s_at | C5orf18 | BC000232 | Chromosome 5 open reading frame 18 |
| 78 | 218534_s_at | VG5Q | NM_018046 | Angiogenic factor VG5Q |
| 79 | 220425_x_at | ROPN1 | NM_017578 | Ropporin, rhophilin associated protein 1 |
| 80 | 208788_at | ELOVL5 | AL136939 | ELOVL family member 5, elongation of long chain fatty acids (FEN1/Elo2, SUR4/Elo3) |
| 81 | 212501_at | CEBPB | AL564683 | CCAAT/enhancer binding protein (C/EBP), β |
| 82 | 51158_at | LOC400451 | AI801973 | Hypothetical gene supported by AK075564; BC060873 |
| 83 | 204284_at | PPP1R3C | N26005 | Protein phosphatase 1, regulatory (inhibitor) subunit 3C |
| 84 | 219918_s_at | ASPM | NM_018123 | asp (abnormal spindle)-like, microcephaly associated (Drosophila) |
| 85 | 211063_s_at | NCK1 | BC006403 | NCK adaptor protein 1 |
| 86 | 221834_at | LONP | AV700132 | Peroxisomal Ion protease |
| 87 | 218195_at | C6orf211 | NM_024573 | Chromosome 6 open reading frame 211 |
| 88 | 207571_x_at | C1orf38 | NM_004848 | Chromosome 1 open reading frame 38 |
| 89 | 216988_s_at | PTP4A2 | L48722 | Protein tyrosine phosphatase type IVA member 2 |
| 90 | 204667_at | FOXA1 | NM_004496 | Forkhead box A1 |
| 91 | 218854_at | SART2 | NM_013352 | Squamous cell carcinoma antigen recognized by T-cells 2 |
| 92 | 200719_at | SKP1A | BE964043 | S-phase kinase-associated protein 1A (p19A) |
| 93 | 201754_at | COX6C | NM_004374 | Cytochrome c oxidase subunit VIc |
| 94 | 213651_at | PIB5PA | AI935720 | Phosphatidylinositol (4,5) bisphosphate 5-phosphatase, A |
| 95 | 218104_at | TEX10 | NM_017746 | Testis expressed sequence 10 |
| 96 | 209870_s_at | APBA2 | AB014719 | Amyloid β (A4) precursor protein-binding, family A, member 2 (X11-like) |
| 97 | 204567_s_at | ABCG1 | NM_004915 | ATP-binding cassette, sub-family G (WHITE), member 1 |
| 98 | 210397_at | DEFB1 | U73945 | Defensin, β1 |
| 99 | 219615_s_at | KCNK5 | NM_003740 | Potassium channel, subfamily K, member 5 |
| 100 | 202089_s_at | SLC39A6 | NM_012319 | Solute carrier family 39 (zinc transporter), member 6 |
| 101 | 214431_at | GMPS | NM_003875 | Guanine monophosphate synthetase |
| 102 | 60471_at | RIN3 | AA625133 | Ras and Rab interactor 3 |
| 103 | 209324_s_at | RGS16 | BF304996 | Regulator of G-protein signalling 16 |
| 104 | 218532_s_at | FLJ20152 | NM_019000 | Hypothetical protein FLJ20152 |
| 105 | 205081_at | CRIP1 | NM_001311 | Cysteine-rich protein 1 (intestinal) |
| 106 | 218239_s_at | GTPBP4 | NM_012341 | GTP binding protein 4 |
| 107 | 215329_s_at | SLC35E2 | AL031282 | Solute carrier family 35, member E2 |
| 108 | 217979_at | TM4SF13 | NM_014399 | Transmembrane 4 superfamily member 13 |
| 109 | 218966_at | MYO5C | NM_018728 | Myosin VC |
| 110 | 203773_x_at | BLVRA | NM_000712 | Biliverdin reductase A |
| 111 | 203287_at | LAD1 | NM_005558 | Ladinin 1 |
| 112 | 206249_at | MAP3K13 | NM_004721 | Mitogen-activated protein kinase kinase kinase 13 |
| 113 | 200824_at | GSTP1 | NM_000852 | Glutathione S-transferase π |
| 114 | 208358_s_at | UGT8 | NM_003360 | UDP glycosyltransferase 8 (UDP-galactose ceramide galactosyltransferase) |
| 115 | 203702_s_at | TTLL4 | AL043927 | Tubulin tyrosine ligase-like family, member 4 |
| 116 | 52940_at | SIGIRR | AA085764 | Signal Ig IL-1R-related molecule |
| 117 | 203384_s_at | GOLGA1 | NM_002077 | Golgi autoantigen, golgin subfamily a, 1 |
| 118 | 218236_s_at | PRKD3 | NM_005813 | Protein kinase D3 |
| 119 | 218483_s_at | FLJ21827 | NM_020153 | Hypothetical protein FLJ21827 |
| 120 | 201980_s_at | RSU1 | NM_012425 | Ras suppressor protein 1 |
| 121 | 203682_s_at | IVD | NM_002225 | Isovaleryl Coenzyme A dehydrogenase |
| 122 | 205363_at | BBOX1 | NM_003986 | Butyrobetaine (γ), 2-oxoglutarate dioxygenase (γ butyrobetaine) |
| 123 | 205471_s_at | DACH1 | NM_004392 | Dachshund homolog 1 (Drosophila) |
| 124 | 218931_at | RAB17 | NM_022449 | RAB17, member RAS oncogene family |
| 125 | 204751_x_at | DSC2 | NM_004949 | Desmocollin 2 |
| 126 | 204881_s_at | UGCG | NM_003358 | UDP-glucose ceramide glucosyltransferase |
| 127 | 205300_s_at | U1SNRNPBP | NM_022717 | U11/U12 snRNP 35K |
| 128 | 201833_at | HDAC2 | NM_001527 | histone deacetylase 2 |
| 129 | 219100_at | OBFC1 | NM_024928 | oligonucleotide/oligosaccharide-binding fold containing 1 |
| 130 | 209531_at | GSTZ1 | BC001453 | glutathione transferase zeta 1 (maleylacetoacetate isomerase) |
| 131 | 217929_s_at | PKD1-like | NM_024874 | polycystic kidney disease 1-like |
| 132 | 217838_s_at | EVL | NM_016337 | Enah/Vasp-like |
| 133 | 201300_s_at | PRNP | NM_000311 | prion protein (p27-30) |
| 134 | 219212_at | HSPA14 | NM_016299 | heat shock 70 kDa protein 14 |
| 135 | 221641_s_at | ACATE2 | AF241787 | likely ortholog of mouse acyl-Coenzyme A thioesterase 2, mitochondrial |
| 136 | 213260_at | FOXC1 | AU145890 | forkhead box C1 |
| 137 | 201564_s_at | FSCN1 | NM_003088 | fascin homolog 1, actin-bundling protein (Strongylocentrotus purpuratus) |
| 138 | 217823_s_at | UBE2J1 | AF151039 | ubiquitin-conjugating enzyme E2, J1 (UBC6 homolog, yeast) |
| 139 | 220173_at | C14orf45 | NM_025057 | chromosome 14 open reading frame 45 |
| 140 | 202320_at | GTF3C1 | NM_001520 | general transcription factor IIIC, polypeptide 1, α 220 kDa |
| 141 | 220658_s_at | ARNTL2 | NM_020183 | aryl hydrocarbon receptor nuclear translocator-like 2 |
| 142 | 202207_at | ARL7 | BG435404 | ADP-ribosylation factor-like 7 |
| 143 | 209396_s_at | CHI3L1 | M80927 | chitinase 3-like 1 (cartilage glycoprotein-39) |
| 144 | 222011_s_at | TCP1 | BF224073 | t-complex 1 |
| 145 | 219686_at | STK32B | NM_018401 | serine/threonine kinase 32B |
| 146 | 212314_at | KIAA0746 | AB018289 | KIAA0746 protein |
| 147 | 209494_s_at | ZNF278 | AI807017 | zinc finger protein 278 |
| 148 | 219806_s_at | FN5 | NM_020179 | FN5 protein |
| 149 | 204688_at | SGCE | NM_003919 | sarcoglycan, epsilon |
| 150 | 201636_at | NA | BG025078 | Homo sapiens cDNA clone IMAGE: 4364070 |
| 151 | 209025_s_at | SYNCRIP | AF037448 | synaptotagmin binding, cytoplasmic RNA interacting protein |
| 152 | 201915_at | SEC63 | NM_007214 | SEC63-like (S. cerevisiae) |
| 153 | 219889_at | FRAT1 | NM_005479 | frequently rearranged in advanced T-cell lymphomas |
| 154 | 210942_s_at | SIAT10 | AB022918 | sialyltransferase 10 (α-2,3-sialyltransferase VI) |
| 155 | 208103_s_at | ANP32E | NM_030920 | acidic (leucine-rich) nuclear phosphoprotein 32 family, member E |
| 156 | 212780_at | SOS1 | AA700167 | son of sevenless homolog 1 (Drosophila) |
| 157 | 219010_at | FLJ10901 | NM_018265 | hypothetical protein FLJ10901 |
| 158 | 202121_s_at | BC-2 | NM_014453 | putative breast adenocarcinoma marker (32 kD) |
| 159 | 205109_s_at | ARHGEF4 | NM_015320 | Rho guanine nucleotide exchange factor (GEF) 4 |
| 160 | 209631_s_at | GPR37 | U87460 | G protein-coupled receptor 37 (endothelin receptor type B-like) |
| 161 | 212846_at | KIAA0179 | AA811192 | KIAA0179 |
| 162 | 213419_at | APBB2 | U62325 | amyloid β (A4) precursor protein-binding, family B, member 2 |
| 163 | 210466_s_at | PAI-RBPI | BC002488 | PAI-1 mRNA-binding protein |
| 164 | 201407_s_at | PPP1CB | AI186712 | protein phosphatase 1, catalytic subunit, β isoform |
| 165 | 218618_s_at | FAD104 | NM_022763 | factor for adipocyte differentiation 104 |
| 166 | 220533_at | FLJ13385 | NM_024853 | hypothetical protein FLJ13385 |
| 167 | 206364_at | KIF14 | NM_014875 | kinesin family member 14 |
| 168 | 210886_x_at | TP53AP1 | AB007457 | TP53 activated protein 1 |
| 169 | 210319_x_at | MSX2 | D89377 | msh homeobox homolog 2 (Drosophila) |
| 170 | 206565_x_at | SMA3 | NM_006780 | SMA3 |
| 171 | 221562_s_at | SIRT3 | AF083108 | sirtuin (silent mating type information regulation 2 homolog) 3 (S. cerevisiae) |
| 172 | 218489_s_at | ALAD | BC000977 | aminolevulinate, delta-, dehydratase |
| 173 | 215723_s_at | PLD1 | AJ276230 | phospholipase D1, phosphatidylcholine-specific |
| 174 | 212759_s_at | TCF7L2 | AI703074 | transcription factor 7-like 2 (T-cell specific, HMG-box) |
| 175 | 209173_at | AGR2 | AF088867 | anterior gradient 2 homolog (Xenopus laevis) |
| 176 | 201215_at | RAB26 | NM_014353 | RAB26, member RAS oncogene family |
| 177 | 219562_at | PLS3 | NM_005032 | plastin 3 (T isoform) |
| 178 | 209170_s_at | GPM6B | AI419030 | glycoprotein M6B |
| 179 | 209745_at | COQ7 | AK024291 | coenzyme Q7 homolog, ubiquinone (yeast) |
| 180 | 211110_s_at | AR | AF162704 | androgen receptor (dihydrotestosterone receptor; testicular feminization) |
| 181 | 212508_at | MOAP1 | AK024029 | modulator of apoptosis 1 |
| 182 | 201012_at | ANXA1 | NM_000700 | annexin A1 |
| 183 | 204785_x_at | IFNAR2 | L41944 | interferon (α, β and omega) receptor 2 |
| 184 | 218440_at | MCCC1 | NM_020166 | methylcrotonoyl-Coenzyme A carboxylase 1 (α) |
| 185 | 219861_at | FLJ10634 | NM_018163 | hypothetical protein FLJ10634 |
| 186 | 205429_s_at | MPP6 | NM_016447 | membrane protein, palmitoylated 6 (MAGUK p55 subfamily member 6) |
| 187 | 217028_at | CXCR4 | AJ224869 | chemokine (C—X—C motif) receptor 4 |
| 188 | 210687_at | CPT1A | BC000185 | carnitine palmitoyltransferase 1A (liver) |
| 189 | 202772_at | HMGCL | NM_000191 | 3-hydroxymethyl-3-methylglutaryl-Coenzyme A lyase |
| 190 | 212442_s_at | LASS6 | BG289001 | LAG1 longevity assurance homolog 6 (S. cerevisiae) |
| 191 | 220432_s_at | CYP39A1 | NM_016593 | cytochrome P450, family 39, subfamily A, polypeptide 1 |
| 192 | 202146_at | IFRD1 | AA747426 | interferon-related developmental regulator 1 |
| 193 | 200790_at | ODC1 | NM_002539 | ornithine decarboxylase 1 |
| 194 | 210648_x_at | SNX3 | AB047360 | sorting nexin 3 |
| 195 | 205996_s_at | AK2 | NM_013411 | adenylate kinase 2 |
| 196 | 212462_at | MYST4 | AU144267 | MYST histone acetyltransferase (monocytic leukemia) 4 |
| 197 | 214806_at | BICD1 | U90030 | bicaudal D homolog 1 (Drosophila) |

### REFERENCES

U.S. Pat. Nos. 5,445,934; 5,532,128; 5,556,752; 5,242,974; 5,384,261; 5,405,783; 5,412,087; 5,424,186; 5,429,807; 5,436,327; 5,472,672; 5,527,681; 5,529,756; 5,545,531; 5,554,501; 5,561,071; 5,571,639; 5,593,839; 5,599,695; 5,624,711; 5,658,734; 5,700,637; 6,004,755; 6,136,182; 6,218,114; 6,218,122; and 6,271,002
US patent publication: 20030194734
Akhurst et al. (2001) Trends Cell Biol. 11:S44-51.
Ammanamanchi et al. (2004) J Biol Chem 279:32620-5.
Binder et al. (1985) J Cell Biol. 101:1371-8.
Chen et al. (2005) J Biol Chem. 280:4632-4638
Eckert et al. (2004) Cancer Res. 64:4585-92.
Emmert-Buck et al. (1996) Science 274:998-1001.
Erlander et al. (2004) WO 2004/07014.
Fernando et al. (2004) Mol. Biol. Cell 15:3266-84.
Foekens et al. (1989) Cancer Res. 49:5823-8.
Fox et al. (2004) Biochem. Biophys. Res. Commun. 318:882-92.
Golub et al. (1999) “Molecular classification of cancer: class discovery and class prediction by gene expression monitoring” Science 286:531-537
Gruvberger et al. (2001) Cancer Res. 61:5979-84.
Hall et al. (2001) J Biol Chem. 276:36869-72.
Kamme et al. (2004) Methods Mol. Med. 99:215-23.
Khoo et al. (1997) Protein Exp. Purif. 9:379-87.
Komuro et al. (2004) Oncogene 23:6914-23.
Luo et al. (1999) Nat. Med. 5:117-22.
Lutfalla et al. (1995) EMBO J. 14:5100-8.
Luzzi et al. (2003) J. Mol. Diagn. 5:9-14.
Ma et al. (2003) Proc. Natl. Acad. Sci. USA 100:5974-9.
Malbert-Colas et al. (2003) Pflugers Arch. 447:35-43.
Mandlekar et al. (2001) Apoptosis 6:469-77.
Marinkovich et al. (1996) J Invest. Dermatol. 106:734-8.
Matsui et al. (2003) Anticancer Res. 23:195-200.
Moggs et al. (2001) EMBO Rep. 2:775-81.
Mokbel (2003) Curr. Med. Res. Opin. 19:683-8.
Nakamura et al. (2004) Oncogene 23:2385-400.
Nishidate et al. (2004) Int. J. Oncol. 25:797-819.
Parl (2000) “Estrogens, estrogen receptor and breast cancer” IOS Press/Ohmsha Amsterdam
Polacek et al. (2003) Physiol. Genomics 13:147-56.
Poliakov et al. (2004) Dev. Cell 7:465-80.
Ramaswamy et al. (2001) “Multiclass cancer diagnosis using tumor gene expression signatures” Proc Natl Acad Sci USA 98:15149-15154
Seth et al. (2003) Anticancer Res. 23:2043-51.
Simstein et al. (2003) Exp. Biol. Med. 228:995-1003.
Sotiriou et al. (2003) Proc. Natl. Acad. Sci. USA 100: 10393-8.
Spillantini et al. (1998) Trends Neurosci. 21:428-33.
Su et al. (2001) “Molecular classification of human carcinomas by use of gene expression signatures” Cancer Res 61:7388-7393
Sun et al. (2005) Exp. Cell Res. 302:96-107.
Tamimi et al. (2004) Invest. Opthalmol. Vis. Sci. 45:3904-13.
van't Veer et al. (2002) “Gene expression profiling predicts clinical outcome of breast cancer” Nature West et al. (2001) Proc. Natl. Acad. Sci. USA 98:11462-7.
Wu et al. (2003) J. Biol. Chem. 278:15192-200.
Wu et al. (2003) J. Biol. Chem. 278:51673-84.
Yim et al. (2003) Toxicol. Pathol. 31:295-303.

1. A method of determining estrogen receptor expression status comprising the steps of
a. obtaining a bulk tissue tumor sample from a breast cancer patient; and
b. measuring the expression levels in the sample of genes selected from the group consisting of those encoding mRNA:
i. corresponding to SEQ ID Nos listed in Table 2 or 3; or
ii. recognized by the probe sets selected from the group consisting of psids corresponding to SEQ ID Nos listed in Table 2 or 3
wherein the gene expression levels above or below pre-determined cut-off levels are indicative of estrogen receptor expression status.

2. A method of determining estrogen receptor expression status comprising the steps of
a. obtaining a microscopically isolated tumor sample from a breast cancer patient; and
b. measuring the expression levels in the sample of genes selected from the group consisting of those encoding mRNA:
i. corresponding to SEQ ID Nos listed in Table 2 or 4; or
ii. recognized by the probe sets selected from the group consisting of psids corresponding to SEQ ID Nos listed in Table 2 or 4
wherein the gene expression levels above or below pre-determined cut-off levels are indicative of estrogen receptor expression status.

3. A method of determining breast cancer patient treatment protocol comprising the steps of
a. obtaining a bulk tissue tumor sample from a breast cancer patient; and
b. measuring the expression levels in the sample of genes selected from the group consisting of those encoding mRNA:
i. corresponding to SEQ ID Nos listed in Table 2 or 3; or
ii. recognized by the probe sets selected from the group consisting of psids corresponding to SEQ ID Nos listed in Table 2 or 3
wherein the gene expression levels above or below pre-determined cut-off levels are sufficiently indicative of risk of recurrence to enable a physician to determine the degree and type of therapy recommended to prevent recurrence.

4. A method of determining breast cancer patient treatment protocol comprising the steps of
a. obtaining a microscopically isolated tumor sample from a breast cancer patient; and
b. measuring the expression levels in the sample of genes selected from the group consisting of those encoding mRNA:
i. corresponding to SEQ ID Nos listed in Table 2 or 4; or
ii. recognized by the probe sets selected from the group consisting of psids corresponding to SEQ ID Nos listed in Table 2 or 4
wherein the gene expression levels above or below pre-determined cut-off levels are sufficiently indicative of risk of recurrence to enable a physician to determine the degree and type of therapy recommended to prevent recurrence.

5. A method of treating a breast cancer patient comprising the steps of:
a. obtaining a bulk tissue tumor sample from a breast cancer patient; and
b. measuring the expression levels in the sample of genes selected from the group consisting of those encoding mRNA:
i. corresponding to SEQ ID Nos listed in Table 2 or 3; or
ii. recognized by the probe sets selected from the group consisting of psids corresponding to SEQ ID Nos listed in Table 2 or 3 and;
c. treating the patient with adjuvant therapy if they are a high risk patient.

6. A method of treating a breast cancer patient comprising the steps of:
a. obtaining a microscopically isolated tumor sample from a breast cancer patient; and
b. measuring the expression levels in the sample of genes selected from the group consisting of those encoding mRNA:
i. corresponding to SEQ ID Nos listed in Table 2 or 4; or
ii. recognized by the probe sets selected from the group consisting of psids corresponding to SEQ ID Nos listed in Table 2 or 4 and;
c. treating the patient with adjuvant therapy if they are a high risk patient.

7. The method of any one of claims 1-6 wherein the sample is obtained from a primary tumor.

8. The method of claim 1, 3 or 5 wherein the bulk tissue preparation is obtained from a biopsy or a surgical specimen.

9. The method of claim 2, 4 or 6 wherein the microscopic isolation is by laser capture microdissection.

10. The method of any one of claims 1-6 further comprising measuring the expression level of at least one gene constitutively expressed in the sample.

11. The method of any one of claims 1-6 wherein the specificity is at least about 40%.

12. The method of any one of claims 1-6 wherein the sensitivity is at least at least about 90%.

13. The method of any one of claims 1-6 wherein the expression pattern of the genes is compared to an expression pattern indicative of a relapse patient.

14. The method of claim 13 wherein the comparison of expression patterns is conducted with pattern recognition methods.

15. The method of claim 14 wherein the pattern recognition methods include the use of a Cox's proportional hazards analysis.

16. The method of any one of claims 1-6 wherein the pre-determined cut-off levels are at least 1.5-fold over- or under-expression in the sample relative to benign cells or normal tissue.

17. The method of any one of claims 1-6 wherein the pre-determined cut-off levels have at least a statistically significant p-value over- or under-expression in the sample having metastatic cells relative to benign cells or normal tissue.

18. The method of claim 17 wherein the p-value is less than 0.05.

19. The method of any one of claims 1-6 wherein gene expression is measured on a microarray or gene chip.

20. The method of claim 19 wherein the microarray is a cDNA array or an oligonucleotide array.

21. The method of claim 20 wherein the microarray or gene chip further comprises one or more internal control reagents.

22. The method of any one of claims 1-6 wherein gene expression is determined by nucleic acid amplification conducted by polymerase chain reaction (PCR) of RNA extracted from the sample.

23. The method of claim 22 wherein said PCR is reverse transcription polymerase chain reaction (RT-PCR).

24. The method of claim 23, wherein the RT-PCR further comprises one or more internal control reagents.

25. The method of any one of claims 1-6 wherein gene expression is detected by measuring or detecting a protein encoded by the gene.

26. The method of claim 25 wherein the protein is detected by an antibody specific to the protein.

27. The method of any one of claims 1-6 wherein gene expression is detected by measuring a characteristic of the gene.

28. The method of claim 27 wherein the characteristic measured is selected from the group consisting of DNA amplification, methylation, mutation and allelic variation.

29. A composition comprising at least one probe set selected from the group consisting of the SEQ ID NOs: listed in Table 2, 3 and/or 4.

30. A kit for conducting an assay to determine estrogen receptor expression status a biological sample comprising: materials for detecting isolated nucleic acid sequences, their complements, or portions thereof of a combination of genes selected from the group consisting of those encoding mRNA corresponding to the SEQ ID NOs: listed in Table 2, 3 and/or 4.

31. The kit of claim 30 wherein the SEQ ID NOs. are those in Table 2 and/or 3.

32. The kit of claim 30 wherein the SEQ ID NOs. are listed in Table 2 and/or 4.

33. The kit of claim 30 further comprising reagents for conducting a microarray analysis.

34. The kit of claim 30 further comprising a medium through which said nucleic acid sequences, their complements, or portions thereof are assayed.

35. Articles for assessing breast cancer status comprising: materials for detecting isolated nucleic acid sequences, their complements, or portions thereof of a combination of genes selected from the group consisting of those encoding mRNA corresponding to the SEQ ID NOs: listed in Table 2, 3 and/or 4.

36. The articles of claim 35 wherein the SEQ ID NOs. are those in Table 2 and/or 3.

37. The articles of claim 35 wherein the SEQ ID NOs. are listed in Table 2 and/or 4.

38. The articles of claim 35 further comprising reagents for conducting a microarray analysis.

39. The articles of claim 35 further comprising a medium through which said nucleic acid sequences, their complements, or portions thereof are assayed.

40. A microarray or gene chip for performing the method of any one of claims 1-6.

41. The microarray of claim 40 comprising isolated nucleic acid sequences, their complements, or portions thereof of a combination of genes selected from the group consisting of those encoding mRNA corresponding to the SEQ ID NOs: listed in Table 2, 3 and/or 4.

42. The microarray of claim 41 comprising a cDNA array or an oligonucleotide array.

43. The microarray of claim 41 further comprising or more internal control reagents.

44. A diagnostic/prognostic portfolio comprising isolated nucleic acid sequences, their complements, or portions thereof of a combination of genes selected from the group consisting of those encoding mRNA corresponding to the SEQ ID NOs: listed in Table 2, 3 and/or 4.
